(19) United States
(12) Patent Application Publication
(10) Pub. No.: US 2023/0190660 A1
(43) Pub. Date: Jun. 22, 2023

(54) ENCAPSULATED PHARMACEUTICAL COMPOSITIONS, RELATED METHODS OF MAKING, AND RELATED METHODS OF TREATMENT

(71) Applicant: PATH, Seattle, WA (US)

(72) Inventors: Manjari Lal, Redmond, WA (US); Changcheng Zhu, Redmond, WA (US); Thinh Le, Seattle, WA (US)

(73) Assignee: PATH, Seattle, WA (US)

(21) Appl. No.: 18/060,766

(22) Filed: Dec. 1, 2022

Related U.S. Application Data
(60) Provisional application No. 63/285,856, filed on Dec. 3, 2021.

(51) Int. Cl. A61K 9/20; A61K 38/28; A61P 3/10

(52) U.S. Cl. CPC A61K 9/2081; A61K 38/28; A61K 9/205; A61K 9/2018; A61P 3/10; A61K 9/0053

(57) ABSTRACT

Temperature-stable, gastric-resistant formulation suitable for oral and sustained delivery of proteins, including insulin, and for nucleic acids (e.g., DNA, RNA), and related methods of making and use are described. In an embodiment, The thermostable and acid-stable pharmaceutical composition includes an inclusion complex, where the inclusion complex includes a lipophilic active pharmaceutical ingredient-carrier ionic association complex encapsulated in a β-cyclodextrin or derivative thereof. In an embodiment, the pharmaceutical composition ion-pair complex encapsulated in the β-cyclodextrin or derivative thereof is in the form of a convenient dissolvable tablet dosage form.

### CROSS-REFERENCE TO RELATED APPLICATION

This application claims the benefit of U.S. Provisional Application No. 63/285,856, filed Dec. 3, 2021, expressly incorporated herein by reference in its entirety.

### STATEMENT OF GOVERNMENT LICENSE RIGHTS

This invention was made with Government support under W81XWH-19-1-0085 awarded by the Department of Defense. The Government has certain rights in the invention.

### BACKGROUND

Oral administration of drugs, such as peptides and proteins, and vaccines is the gold standard for delivery to patients due to patient acceptance, low cost, and lack of need for peripheral devices. However, parenteral administration remains the main mode of delivery of macromolecule therapeutics and vaccines for a variety of technical issues.

Insulin, a peptide hormone that must be dosed repeatedly, exemplifies the challenges of parenteral administration from the standpoint of patient acceptance as well as the technical challenges to successful oral delivery. Insulin remains underused due to the adherence challenge of frequent injections. Degradation of insulin at both elevated and frozen temperatures makes it difficult to distribute and store in areas with limited or no access to refrigeration and a temperature-controlled supply chain. Oral insulin is attractive for improving patient adherence. However, the high molecular weight and hydrophilicity of insulin limits intestinal absorption, in addition to gastrointestinal degradation due to both low pH in the stomach and the presence of proteolytic enzymes in the stomach and small intestine.

Previous oral insulin approaches using nanoparticles, nanocarriers, vesicles, and emulsions require multiple excipients and processing steps that increase cost. Furthermore, none suggest a heat-stable oral insulin in a product format that is easily manufacturable, accessible due to less stringent storage requirements, or has broader acceptability for all patient populations.

Oral delivery of nucleic acids such as RNA as drugs or vaccines presents similar challenges to oral delivery of peptides and proteins. In the case of RNA molecules, lability stems in part from the 2′-hydroxyl group on the ribose pentose ring that is susceptible to hydrolysis. Inside the body, RNA and DNA are cleaved by the human ribonuclease and deoxyribonuclease, respectively present in the tissues, and even by pepsin, making oral delivery extremely challenging.

### SUMMARY

To address these and related challenges, the present disclosure provides pharmaceutical compositions, related methods of making pharmaceutical compositions, and related methods of treating a subject in need thereof.

In an aspect, the present disclosure provides a pharmaceutical composition. In an embodiment, the pharmaceutical composition comprises an active pharmaceutical ingredient-carrier ionic association complex; and a β-cyclodextrin or derivative thereof, wherein the β-cyclodextrin encapsulates the active pharmaceutical ingredient-carrier ionic association complex to form a β-cyclodextrin-active pharmaceutical ingredient-carrier inclusion complex.

In an embodiment, wherein the β-cyclodextrin derivative comprises a hydroxy propyl β-cyclodextrin. In an embodiment, the β-cyclodextrin or derivative thereof is present in the pharmaceutical composition in an amount of from about 2% to about 10% by weight.

In an embodiment, the active pharmaceutical ingredient is cationic and the carrier is anionic. In an embodiment, the active pharmaceutical ingredient is anionic and the carrier is cationic.

In an embodiment, the active pharmaceutical ingredient comprises a peptide or a protein.

In an embodiment, the active pharmaceutical ingredient comprises insulin.

In an embodiment, the carrier comprises a bile salt. In an embodiment, the bile salt comprises a deoxycholate salt (e.g., sodium deoxycholate, sodium cholate, sodium taurocholate). In an embodiment, the ionic association complex comprises insulin:bile salt at a molar ratio of from 1:7 to 1:28 (e.g., 1:28).

In an embodiment, wherein the active pharmaceutical ingredient comprises an RNA agent and/or a DNA agent.

In an embodiment, the carrier comprises a fatty amine, a cationic polysaccharide (e.g., chitosan), cholesterol-PEG-amine, tocopheryl PEG amine, tocopheryl amine, coconut amine, octyl amine, lauryl amine stearyl amine, oleyl amine, or any combination thereof. In an embodiment, the cationic carrier comprises a fatty amine, cholesterol-PEG-amine, tocopheryl PEG amine, or any combination thereof.

In an embodiment, the active pharmaceutical ingredient-carrier ionic association complex comprises a liposome, a micelle, a vesicle, a solid particle, a colloid, or any combination thereof. In an embodiment, the active pharmaceutical ingredient-cationic carrier ionic association complex comprises a liposome or a micelle.

In an embodiment, the pharmaceutical composition has greater stability to enzymatic degradation compared to an unencapsulated active pharmaceutical ingredient ionic association complex or to a free active pharmaceutical ingredient (i.e., an un-complexed and unencapsulated active pharmaceutical ingredient). In an embodiment, the active pharmaceutical ingredient has greater stability to enzymatic degradation compared to an unencapsulated active pharmaceutical ingredient ionic association complex or to a free active pharmaceutical ingredient (i.e., an un-complexed and unencapsulated RNA agent and/or DNA agent).

In an embodiment, a molar ratio of the active pharmaceutical ingredient to cationic carrier is from about 1:4 to about 1:30.

In an embodiment, the pharmaceutical composition comprises an oral dosage form. In an embodiment, the pharmaceutical composition comprises a solid oral dosage form, a liquid oral dosage form, or a semi-solid oral dosage form. In an embodiment, the pharmaceutical composition is in the form of a tablet, a capsule, a sachet, a powder, a granule, or an orally dispersible film. In an embodiment, the pharmaceutical composition is in the form of a freeze-dried tablet. In an embodiment, the pharmaceutical composition comprises an injectable dosage form. In an embodiment, the pharmaceutical composition comprises a suspension.

In an embodiment, the ionic association complex is lipophilic.

In an embodiment, the pharmaceutical composition has a sustained release of the active pharmaceutical ingredient at least 40% of a Cₘₐₓ in plasma over 12 hours.

In an embodiment, the pharmaceutical composition is stable at a pH of less than 7.

In an embodiment, the pharmaceutical composition is absorbable in the intestines, when ingested by a mammalian subject.

In an embodiment, the pharmaceutical composition is at least 90% by weight unchanged (i.e., is at least 90% stable) at a temperature of 30° C. and 65% relative humidity for at least 12 weeks and/or at a temperature of 40° C. and 75% relative humidity for at least 8 weeks.

In another aspect, the present disclosure provides a method of forming a pharmaceutical composition of any one of the preceding claims. In an embodiment, the method comprises mixing an amount of the active pharmaceutical ingredient and an amount of the carrier in an aqueous medium (e.g., water) to provide the active pharmaceutical ingredient-carrier ionic association complex; adding the β-cyclodextrin or derivative thereof to the active pharmaceutical ingredient-carrier ionic association complex to provide the β-cyclodextrin-active pharmaceutical ingredient-carrier ionic inclusion complex.

In an embodiment, the method further comprises forming the pharmaceutical composition into an oral dosage form. In an embodiment, forming the pharmaceutical composition into an oral dosage form comprises freeze-drying the pharmaceutical composition.

In another aspect, the present disclosure provides a method of treating a subject having diabetes. In an embodiment, the method comprises orally administrating a therapeutically effective amount of a pharmaceutical composition of any embodiment of the present disclosure including insulin as an active pharmaceutical ingredient.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the subject matter of the present disclosure will become more readily appreciated as the same become better understood by reference to the following detailed description, when taken in conjunction with the accompanying drawings, wherein:

FIGS. 1A and 1B are illustrations of pharmaceutical compositions according to embodiments of the present disclosure;

FIG. 2 schematically illustrates (left) conventional insulin in the stomach and (right) a pharmaceutical composition according to an embodiment of the present disclosure including insulin in the stomach;

FIG. 3A graphically illustrates thermal stability of control insulin and freeze-dried oral insulin tablets according to embodiments of the present disclosure at 2° C.-8° C.;

FIG. 3B graphically illustrates thermal stability of control insulin and freeze-dried oral insulin tablets according to embodiments of the present disclosure at 30° C. and 65% relative humidity (RH);

FIG. 3C graphically illustrates thermal stability of control insulin and an oral insulin tablet according to an embodiment of the present disclosure at 40° C. and 75% RH;

FIG. 4 graphically illustrates glucose blood levels in rats after administration of control insulin and pharmaceutical compositions according to embodiments of the present disclosure;

FIG. 5A schematically illustrates a method of manufacturing a pharmaceutical composition according to an embodiment of the present disclosure;

FIG. 5B schematically illustrates a method of manufacturing a pharmaceutical composition according to an embodiment of the present disclosure;

FIG. 6 provides images of gel electrophoresis of RNA formulations including encapsulated lipoplex siRNA formulations according to embodiments of the present disclosure;

FIG. 7 is an image of gel electrophoresis of various RNA formulations including encapsulated lipoplex siRNA according to embodiments of the present disclosure, which show better protection from RNAse (FBS) degradation compared to naked siRNA;

FIG. 8 is a calibration curve for insulin comparing insulin (IU) and peak area (mAU*min), according to an embodiment of the present disclosure;

FIG. 9 graphically illustrates insulin degradation at various pH levels, according to an embodiment of the present disclosure;

FIGS. 10A-10C provide chemical structures of carriers according to embodiments of the present disclosure;

FIGS. 11A-11C are images of freeze-dried tablets comprising resistant polymers guar gum (FIG. 11A), pullulan (FIG. 11B), and xanthan gum (FIG. 11C), according to embodiments of the present disclosure;

FIG. 12A is an illustration of a capsule for oral delivery and corresponding size scale, according to an embodiment of the present disclosure;

FIG. 12B is an illustration of a freeze-dried tablet for oral delivery and corresponding size scale, according to an embodiment of the present disclosure;

FIG. 13A graphically illustrates in vitro evaluation of control insulin ion-pair formulations including sodium oleate according to embodiments of the present disclosure after incubation in simulated gastrointestinal fluids for 30 minutes at 37° C.;

FIG. 13B graphically illustrates in vitro evaluation of control insulin ion-pair formulations including sodium deoxycholate according to embodiments of the present disclosure after incubation in simulated gastrointestinal fluids for 30 minutes at 37° C.;

FIGS. 14A-14D graphically illustrate blood glucose levels in vitro after administration of subcutaneous insulin and oral tablets at various dosages according to embodiments of the present disclosure at 0 hours (FIG. 14A), 1 hour (FIG. 14B), 4 hours (FIG. 14C), and 8 hours (FIG. 14D) after administration;

FIGS. 15A-15D graphically illustrate blood glucose levels in vitro after administration of subcutaneous insulin and oral tablets at a dosage of 26 IU according to embodiments of the present disclosure at 0 hours (FIG. 15A), 1 hour (FIG. 15B), 4 hours (FIG. 15C), and 8 hours (FIG. 15D) after administration;

FIG. 16 graphically illustrates thermostability of an unformulated insulin and a pharmaceutical composition according to an embodiment of the present disclosure over time;

FIG. 17A graphically illustrates blood glucose levels (mg/dL) versus time in rats orally administered a pharmaceutical composition according to an embodiment of the present disclosure and subcutaneous insulin;

FIG. 17B graphically illustrates blood glucose levels (mg/dL) versus time in rats orally administered a pharmaceutical composition according to an embodiment of the present disclosure and subcutaneous insulin; and

FIG. 17C graphically illustrates blood glucose levels (mg/dL) versus time in rats orally administered a pharmaceutical composition according to an embodiment of the present disclosure.

### DETAILED DESCRIPTION

The present disclosure describes a temperature-stable, gastric-resistant formulation suitable for oral and sustained delivery of proteins, including insulin, and for nucleic acids (e.g., DNA, RNA), and related methods of making and use.

### Pharmaceutical Compositions

In an aspect, the present disclosure provides pharmaceutical compositions. In an embodiment, the pharmaceutical compositions comprise an active pharmaceutical ingredient-carrier ionic association complex; and a β-cyclodextrin or derivative thereof. As discussed further herein, in an embodiment, the β-cyclodextrin encapsulates the active pharmaceutical ingredient-carrier ionic association complex to form a β-cyclodextrin-active pharmaceutical ingredient-carrier inclusion complex.

As discussed further herein and without being bound by any particular theory, it is believed that the inclusion properties and the cryoprotectant attributes of β-cyclodextrin in encapsulating and stabilizing the active pharmaceutical ingredient-carrier ionic association complex forms a heat-stable pharmaceutical composition, which, in certain embodiments, is suitable for oral administration. In this regard, the β-cyclodextrin maintains active pharmaceutical ingredient stability, such as without additional excipients or enteric coating. Further, the pharmaceutical composition is shown to have greater stability to enzymatic degradation compared to an unencapsulated active pharmaceutical ingredient ionic association complex or to a free active pharmaceutical ingredient (i.e., an un-complexed and unencapsulated active pharmaceutical ingredient).

As one example, coupling insulin with a bile salt as a lipophilic ion-pair is shown to enhance intestinal transport, where encapsulation of this ion-pair within a β-cyclodextrin (e.g., hydroxy propyl β-cyclodextrin, or “HP-β-CD”) stabilizes insulin, protecting the insulin from temperature and stomach degradation. Furthermore, the cryoprotectant and polymeric properties of HP-β-CD allows freeze drying of insulin directly into a heat-stable tablet dosage form.

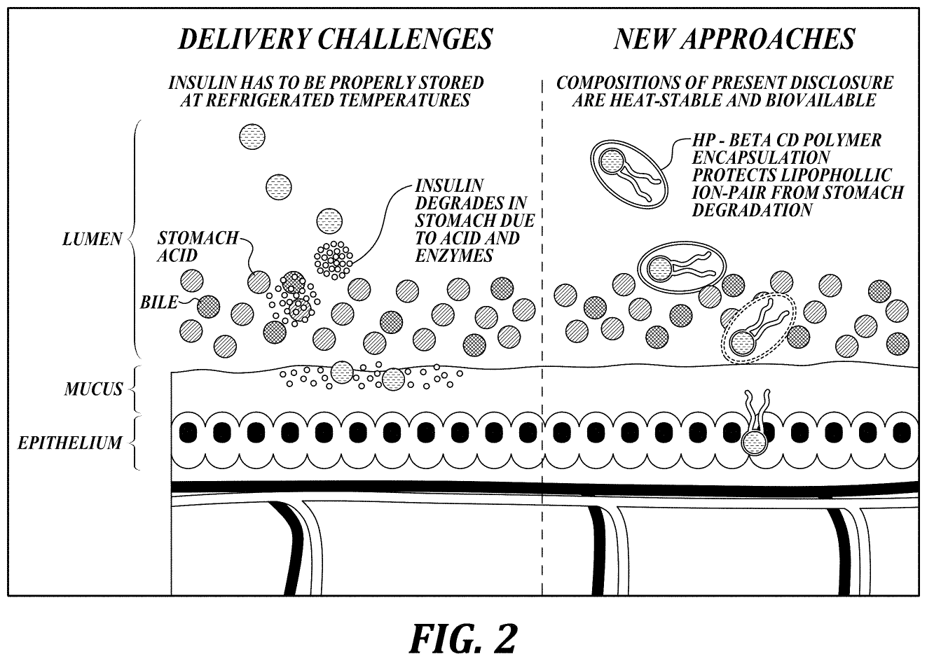

As shown in FIG. 2 (left), insulin degrades in the stomach due to stomach acid and enzymes. Consequently, insulin is poorly absorbed from the gastrointestinal tract.

The right panel of FIG. 2 schematically illustrates a pharmaceutical composition according to an embodiment of the present disclosure including insulin in the stomach. As illustrated, β-cyclodextrin encapsulates the active pharmaceutical ingredient-carrier ionic association complex from stomach degradation. Consequently, the lipophilic ion pairing achieves absorption of the pharmaceutical composition, and, as discussed further herein, provides sustained levels of the active pharmaceutical ingredient. Where the active pharmaceutical ingredient is insulin, the pharmaceutical compositions of the present disclosure also achieve sustained low levels of glucose after administration.

The pharmaceutical compositions of the present disclosure include an active pharmaceutical ingredient-carrier ionic association complex. As discussed further herein, the ionic association complex is a complex formed by ionic interaction between a negatively charged molecule such as a lipophilic molecule (e.g., a lipophilic detergent) with a positively charged molecule, peptide, or protein. In an embodiment, the active pharmaceutical ingredient is cationic, and the carrier is anionic. In another embodiment, the active pharmaceutical ingredient is anionic, and the carrier is cationic.

In an embodiment, the carrier comprises a fatty amine, a cationic polysaccharide (e.g., chitosan), cholesterol-PEG-amine, tocopheryl PEG amine, tocopheryl amine, coconut amine, octyl amine, lauryl amine stearyl amine, oleyl amine, or any combination thereof. In an embodiment, the cationic carrier comprises a fatty amine, cholesterol-PEG-amine, tocopheryl PEG amine, or any combination thereof. In an embodiment, the carrier comprises sodium oleate.

In an embodiment, the carrier comprises a bile salt. In an embodiment, the bile salt comprises a deoxycholate salt. In an embodiment, the deoxycholate salt is chosen from sodium deoxycholate, sodium cholate, sodium taurocholate, and combinations thereof. In an embodiment, the bile salt is sodium cholate hydrate.

In an embodiment, the ionic association complex comprises an active pharmaceutical ingredient:carrier at a molar ratio of from 1:7 to 1:140. In an embodiment, the ionic association complex comprises an active pharmaceutical ingredient:carrier at a molar ratio of from 1:10 to 1:28. In an embodiment, the ionic association complex comprises an active pharmaceutical ingredient:carrier at a molar ratio of from 1:10 to 1:140. In an embodiment, the ionic association complex comprises an active pharmaceutical ingredient:carrier at a molar ratio of from 1:7 to 1:28 (e.g., about 1:28). In an embodiment, the ionic association complex comprises insulin:bile salt at a molar ratio of from 1:7 to 1:28 (e.g., about 1:28). As shown, by decreasing the active pharmaceutical ingredient:carrier ratio, complex efficiency is increased. See Example 2 and TABLE 2. However, by decreasing the active pharmaceutical ingredient:carrier ratio, a coefficient of variation can also decrease. In this regard, a balance can be struck between complexation efficiency and coefficient of variation. Id. Accordingly, an optimal ratio between an active pharmaceutical ingredient and a carrier can be obtained, which can depend on the particular active pharmaceutical ingredient and carrier.

The active pharmaceutical ingredient-carrier ionic association complex can take many forms. In an embodiment, the active pharmaceutical ingredient-carrier ionic association complex comprises a liposome, a micelle, a vesicle, a solid particle, a colloid, or any combination thereof. In an embodiment, the active pharmaceutical ingredient-cationic carrier ionic association complex comprises a liposome or a micelle.

The pharmaceutical compositions of the present disclosure include an active pharmaceutical ingredient. The pharmaceutical compositions of the present disclosure can include many types of active pharmaceutical ingredients. In an embodiment, the active pharmaceutical ingredient is chosen from a nucleic acid, such as an RNA agent or a DNA agent, a peptide, a protein, and combinations thereof. In an embodiment, the active pharmaceutical ingredient comprises insulin.

In an embodiment, the active pharmaceutical ingredient is present in the pharmaceutical composition in a range of about 3% w/w to about 7% w/w. In an embodiment, the active pharmaceutical ingredient is present in the pharmaceutical composition in a range of about 3% w/w to about 6% w/w. In an embodiment, the active pharmaceutical ingredient is present in the pharmaceutical composition in a range of about 3% w/w to about 4% w/w. In an embodiment, the active pharmaceutical ingredient is present in the pharmaceutical composition in a range of about 4% w/w to about 6% w/w. In an embodiment, the active pharmaceutical ingredient is present in the pharmaceutical composition in a range of about 5% w/w to about 6% w/w. In an embodiment, the active pharmaceutical ingredient is about 6% w/w.

In an embodiment, such as where the active pharmaceutical ingredient is an RNA or a DNA agent, the molar ratio of the active pharmaceutical ingredient to cationic carrier is from about 1:4 to about 1:30. In an embodiment, the molar ratio of the active pharmaceutical ingredient to cationic carrier is from about 1:4 to about 1:30. In an embodiment, the molar ratio of the active pharmaceutical ingredient to cationic carrier is from about 1:4 to about 1:15. In an embodiment, the molar ratio of the active pharmaceutical ingredient to cationic carrier is from about 1:10 to about 1:30. In an embodiment, the molar ratio of the active pharmaceutical ingredient to cationic carrier is from about 1:20 to about 1:30.

As above, in an embodiment, the pharmaceutical compositions of the present disclosure include a cyclodextrin. Cyclodextrins are a class of cyclic, non-reducing oligosaccharides built up from six, seven, and eight glucopyranose rings known respectively as alpha, beta, and gamma cyclodextrins. In an embodiment, the cyclodextrin is a β-cyclodextrin. In an embodiment, the cyclodextrin is a cyclodextrin derivative, such as hydroxypropyl-β-cyclodextrin, sulfobutyl ether β-cylcodextrin, randomly methylated β-cyclodextrin, hydroxypropyl-gamma-cyclodextrin, polymerized cyclodextrins, epichlorohydrin-β-cyclodextrin, or carboxy methyl epichlorohydrin β-cyclodextrin. In an embodiment, the β-cyclodextrin is chosen from methyl-β-cyclodextrin, ethyl-β-cyclodextrin, propyl-β-cyclodextrin, and hydroxypropyl β-cyclodextrin. In an embodiment, the β-cyclodextrin derivative comprises a hydroxy propyl β-cyclodextrin.

In an embodiment, the β-cyclodextrin or derivative thereof is present in the pharmaceutical composition in an amount of from about 2% to about 10% by weight. In an embodiment, the β-cyclodextrin or derivative thereof is present in the pharmaceutical composition in an amount of from about 3% to about 9% by weight. In an embodiment, the 3-cyclodextrin or derivative thereof is present in the pharmaceutical composition in an amount of from about 4% to about 8% by weight. In an embodiment, the β-cyclodextrin or derivative thereof is present in the pharmaceutical composition in an amount of from about 5% to about 7% by weight. In an embodiment, the β-cyclodextrin or derivative thereof is present in the pharmaceutical composition in an amount of from about 6% to about 10% by weight. In an embodiment, the β-cyclodextrin or derivative thereof is present in the pharmaceutical composition in an amount of from about 2% to about 5% by weight. In an embodiment, the β-cyclodextrin or derivative thereof is present in the pharmaceutical composition in 1%, 2%, 3%, 4%, 5%, 6%, 7%, 8%, 9%, or 10% by weight.

In an embodiment, the β-cyclodextrin or derivative thereof is present in the pharmaceutical composition in an amount of from about 15% to about 65% by weight. In an embodiment, the β-cyclodextrin or derivative thereof is present in the pharmaceutical composition in an amount of from about 35% to about 65% by weight. In an embodiment, the β-cyclodextrin or derivative thereof is present in the pharmaceutical composition in an amount of from about 35% to about 55% by weight.

While active pharmaceutical ingredient-carrier ionic association complexes are discussed encapsulated by a β-cyclodextrin or derivative thereof to form a β-cyclodextrin-active pharmaceutical ingredient-carrier inclusion complex, it will be understood that the active pharmaceutical ingredient-carrier ionic association complex may form inclusion complexes or otherwise associated with other compounds. As demonstrated in TABLE 6 and Example 3, such inclusion complexes or associations can provide pharmaceutical compositions with high drug recovery from freeze-dried tablets having low coefficients of variability. In an embodiment, the active pharmaceutical ingredient-carrier ionic association complex forms inclusion complexes or is otherwise associated with a compound selected from the group consisting of xanthan gum, guar gum, pullulan, and combinations thereof. In an embodiment, the active pharmaceutical ingredient-carrier ionic association complex forms inclusion complexes or is otherwise associated with pullulan. In an embodiment, the active pharmaceutical ingredient-carrier ionic association complex forms inclusion complexes or is otherwise associated with guar gum.

As discussed further herein, the pharmaceutical compositions can further include additional sugars and polysaccharides. In an embodiment, the pharmaceutical compositions include one or more of sucrose, mannitol, dextran (such as dextran 40), and the like. In an embodiment, such additional sugars are present in a range of 15 w/w % to about 30 w/w %. In an embodiment, such additional sugars are present in a range of 15 w/w % to about 25 w/w %.

In an embodiment, the pharmaceutical compositions of the present disclosure include one or more surfactants. In an embodiment, the pharmaceutical compositions include Tween™, such as Tween 80™. In an embodiment, the one or more surfactants are present in a range of about 2 w/w % to about 3 w/w %.

The pharmaceutical compositions described herein can include other agents, excipients, or stabilizers to improve properties of the composition. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propyl-hydroxybenzoates, talc, magnesium stearate and mineral oil. The formulations can additionally include lubricating agents, wetting agents, emulsifying and suspending agents, preserving agents, sweetening agents or flavoring agents. Examples of emulsifying agents include tocopherol esters such as tocopheryl polyethylene glycol succinate and the like, Pluronic, emulsifiers based on polyoxyethylene compounds, Span 80 and related compounds, and other emulsifiers known in the art and approved for use in animals or human dosage forms. The compositions can be formulated so as to provide rapid, sustained or delayed release of the active ingredient after administration to the patient by employing procedures well known in the art.

The pharmaceutical compositions of the present disclosure can take a number of different forms, suitable for a number of different methods or modes of administration. In an embodiment, the pharmaceutical composition comprises an oral dosage form, such as a freeze-dried or lyophilized oral dosage form.

In some embodiments, the composition is suitable for administration to a human. In some embodiments, the composition is suitable for administration to a mammal, such as, in the veterinary context, including domestic pets and agricultural animals. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, (d) suitable emulsions, and (e) powders. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

As noted herein above and as shown in the Examples of the present disclosure, the pharmaceutical compositions of the present disclosure provide or have a number of advantageous characteristics. Without being limited to any particular theory, it is believed that, in many cases, the advantageous characteristics result at least in part from the β-cyclodextrin encapsulating the active pharmaceutical ingredient-carrier ionic association complex, thereby forming a β-cyclodextrin-active pharmaceutical ingredient-carrier inclusion complex.

In an embodiment, the pharmaceutical composition has a sustained release of the active pharmaceutical ingredient at least 40% of a Cₘₐₓ in plasma over 12 hours.

In an embodiment, the pharmaceutical composition is stable at a pH of less than 7. This advantageously provides stability in acidic environments in vivo, such as in the stomach of a subject. Further, such stability provides that the pharmaceutical composition is absorbable in the intestines, when ingested by a mammalian subject.

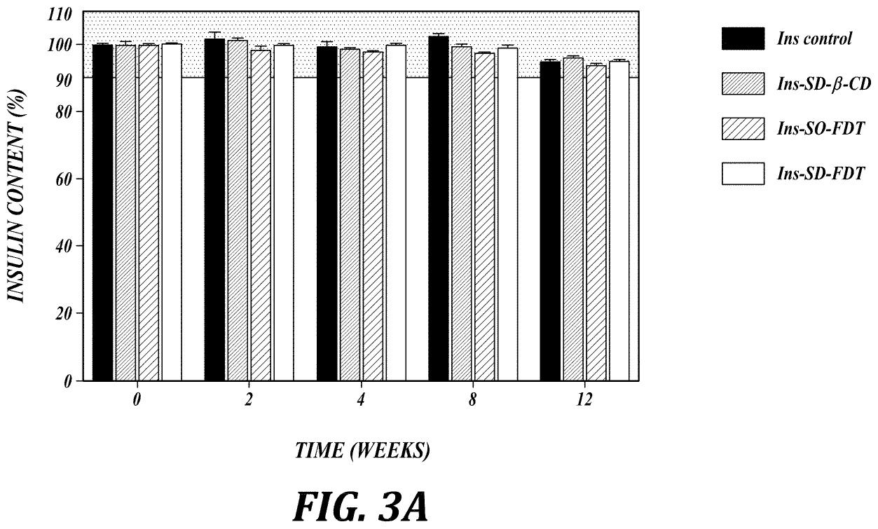

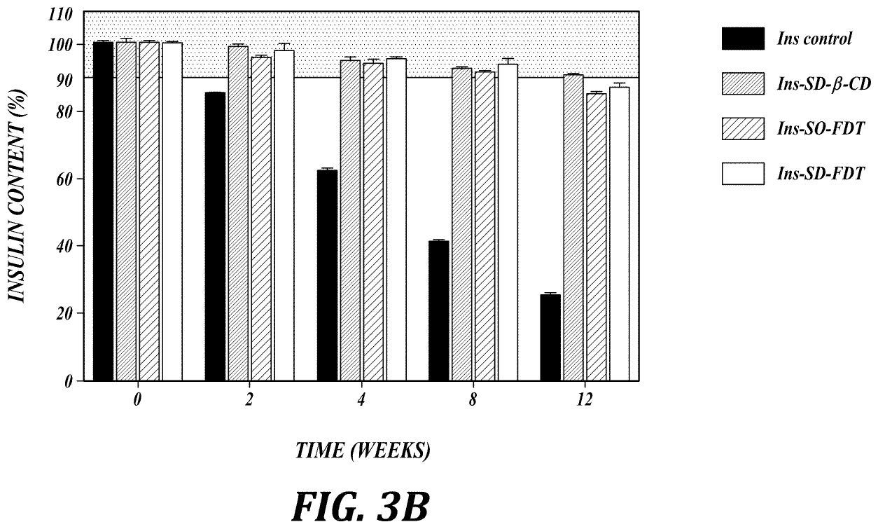

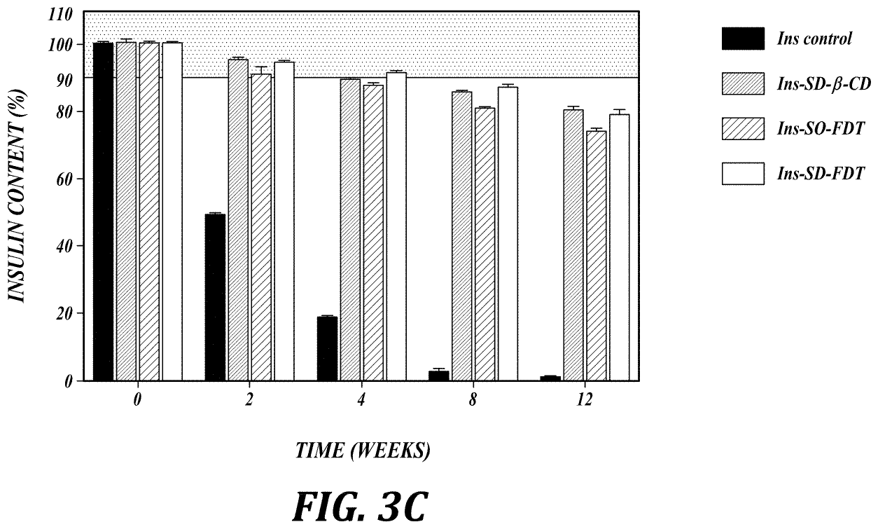

As also discussed further herein and shown in the Examples of the present disclosure, the pharmaceutical compositions of the present disclosure are stable at room temperature and at elevated temperatures. In this regard, the pharmaceutical compositions of the present disclosure may be stored, such as without refrigeration, for extended periods of time with little or no degradation in concentration or efficacy of the active pharmaceutical ingredient. Accordingly, in an embodiment, the pharmaceutical composition is at least 90% by weight unchanged (i.e., is at least 90% stable) at a temperature of 30° C. and 65% relative humidity for at least 12 weeks. Similarly, in an embodiment, the pharmaceutical composition is at least 90% by weight unchanged (i.e., is at least 90% stable) at a temperature of 40° C. and 75% relative humidity for at least 8 weeks. See, for example, FIGS. 3A-3C.

It will be appreciated that in certain embodiments, the pharmaceutical compositions of the present disclosure comprise the components described herein. In certain other embodiments, it will be appreciated that the pharmaceutical compositions of the invention consist essentially of the components described herein, and that in these embodiments the pharmaceutical compositions do not include any additional component that would materially affect the properties of the pharmaceutical compositions (e.g., therapeutic function, effect, or other pharmacokinetic properties). In certain further embodiments, it will be appreciated that the pharmaceutical compositions of the invention consist of the components described herein, and that in these embodiments the pharmaceutical compositions do not include any additional components.

In further aspects, the invention provides articles of manufacture comprising the compositions described herein in suitable packaging. Suitable packaging for compositions described herein are known in the art, and include, for example, vials (such as sealed vials), vessels (such as sealed vessels), ampules, bottles, jars, flexible packaging (such as sealed Mylar or plastic bags), and the like. These articles of manufacture may further be sterilized and/or sealed.

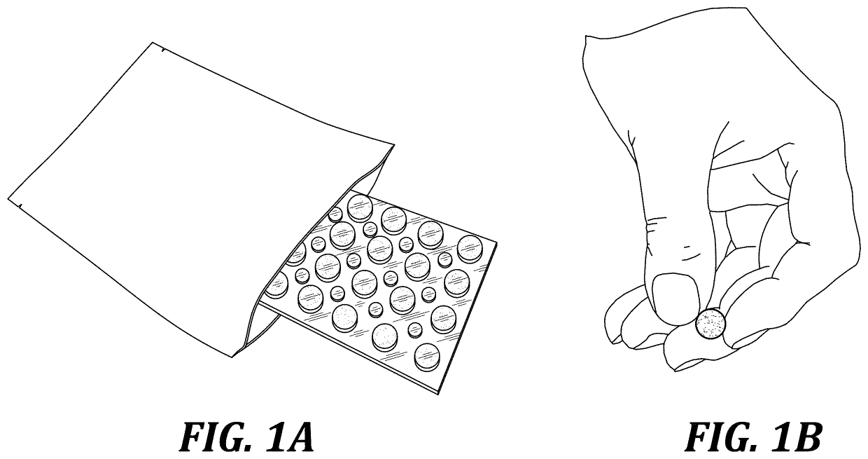

Also provided are unit dosage forms comprising the compositions described herein. These unit dosage forms can be stored in a suitable packaging in single or multiple unit dosages and may also be further sterilized and sealed. In an embodiment, the unit dosage form comprises an amount of the pharmaceutical composition suitable for a single dose of the active ingredient, such as therapeutically effective amount of the active ingredient. In an embodiment, the present disclosure provides a plurality of unit dosage forms each comprising a pharmaceutical composition of the present disclosure, such as in the form of a blister pack wherein each blister of the blister pack comprises a tablet form unit dosage. See, for example, FIG. 1A.

In an embodiment, the pharmaceutical composition comprises a solid oral dosage form, a liquid oral dosage form, or a semi-solid oral dosage form. In an embodiment, the pharmaceutical composition is in the form of a tablet, a capsule, a sachet, a powder, a granule, or an orally dispersible film. See, for example, FIGS. 1A and 1B. In an embodiment, the pharmaceutical composition is in the form of a freeze-dried tablet. In an embodiment, the pharmaceutical composition comprises an injectable dosage form. In an embodiment, the pharmaceutical composition comprises a suspension.

The present invention also provides kits comprising compositions (or unit dosages forms and/or articles of manufacture) described herein and may further comprise instruction(s) on methods of using the composition, such as uses further described herein.

In some embodiments, the kit of the invention comprises the packaging described above. In other embodiments, the kit of the invention comprises the packaging 5 described above and a second packaging comprising a buffer. It may further include other materials desirable from a commercial and user standpoint, including other buffers, diluents, filters, needles, syringes, and package inserts with instructions for performing any methods described herein. Kits may also be provided that contain sufficient dosages of the therapeutic agent as disclosed herein to provide effective treatment for an individual for an extended period, such as any of a week, 2 weeks, 3 weeks, 4 weeks, 6 weeks, 8 weeks, 3 months, 4 months, 5 months, 6 months, 7 months, 8 months, 9 months or more. Kits may also include multiple unit doses of the therapeutic agent and pharmaceutical compositions and instructions for use and packaged in quantities sufficient for storage and use in pharmacies, for example, hospital pharmacies and compounding pharmacies.

### Methods of Making

In another aspect, the present disclosure provides a method of forming a pharmaceutical composition according to any embodiment of the present disclosure.

In an embodiment, the methods of making comprise mixing an amount of the active pharmaceutical ingredient and an amount of the carrier in an aqueous medium to provide the active pharmaceutical ingredient-carrier ionic association complex.

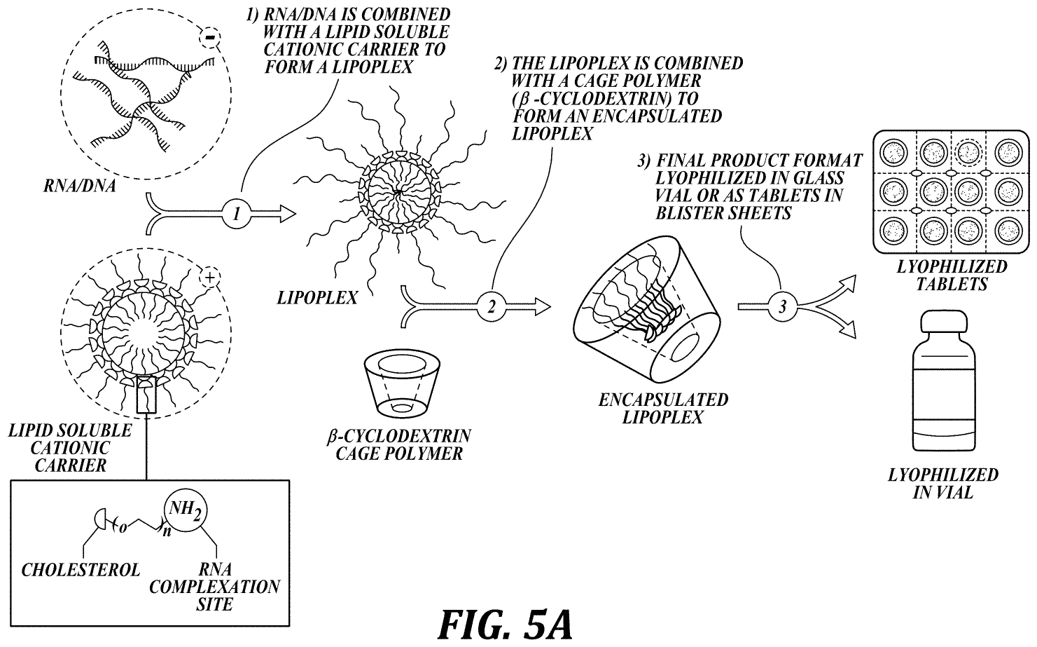

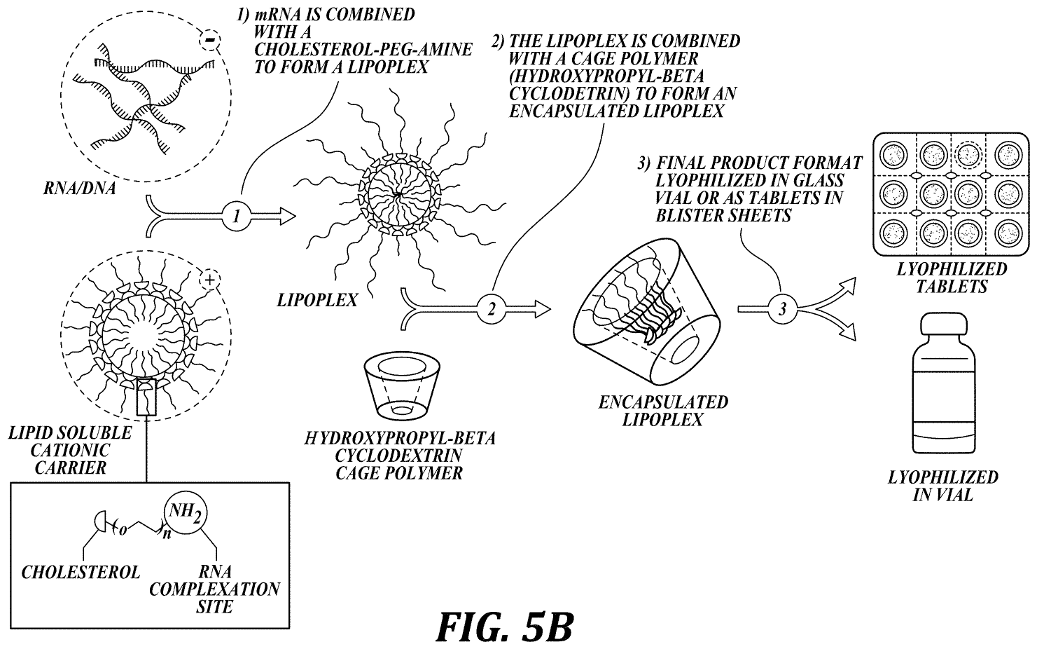

In an embodiment, the active pharmaceutical ingredient and the carrier, when mixed, form an active pharmaceutical ingredient-carrier ionic association complex in the form of a lipoplex. See, for example, FIGS. 5A and 5B. In an embodiment, the active pharmaceutical ingredient-carrier ionic association complex comprises a liposome, a micelle, a vesicle, a solid particle, a colloid, or any combination thereof.

In an embodiment, the active pharmaceutical ingredient is anionic and the carrier is cationic.

In an embodiment, the active pharmaceutical ingredient comprises a peptide or a protein. In an embodiment, the active pharmaceutical ingredient comprises insulin. In an embodiment, the active pharmaceutical ingredient comprises an RNA agent and/or a DNA agent.

In an embodiment, the carrier comprises a bile salt. In an embodiment, the bile salt comprises a deoxycholate salt. In an embodiment, the deoxycholate salt is selected from the group consisting of sodium deoxycholate, sodium cholate, and sodium taurocholate.

In an embodiment, the carrier comprises a fatty amine, a cationic polysaccharide, cholesterol-PEG-amine, tocopheryl PEG amine, tocopheryl amine, coconut amine, octyl amine, lauryl amine stearyl amine, oleyl amine, or any combination thereof.

In an embodiment, the ionic association complex comprises active pharmaceutical ingredient:carrier at a molar ratio of from 1:7 to 1:28. In an embodiment, the molar ratio of the active pharmaceutical ingredient to cationic carrier is from about 1:4 to about 1:30.

In an embodiment, the method further includes adding a cage polymer to the active pharmaceutical ingredient-carrier ionic association complex to provide the pharmaceutical composition. In an embodiment, the cage polymer is a β-cyclodextrin or derivative thereof, which provides a β-cyclodextrin-active pharmaceutical ingredient-carrier ionic inclusion complex. In an embodiment, the β-cyclodextrin derivative comprises a hydroxy propyl β-cyclodextrin. In an embodiment, the β-cyclodextrin or derivative thereof is present in the pharmaceutical composition in an amount of from about 2% to about 10% by weight.

In an embodiment, the method further comprising forming the pharmaceutical composition into an oral dosage form. As described further herein, in an embodiment forming the pharmaceutical composition into an oral dosage form comprises lyophilizing or freeze drying a solution or suspension of the pharmaceutical composition to provide the oral dosage form, such as in a freeze-dried tablet. In an embodiment, forming the pharmaceutical composition into an oral dosage form comprises freeze-drying the pharmaceutical composition. Example lyophilization conditions are described herein and are well known in the art.

### Methods of Treatment

In another aspect, the present disclosure provides a method of treating a subject having diabetes, comprising orally administrating a therapeutically effective amount of a pharmaceutical composition of any embodiment of the present disclosure in which the active pharmaceutical composition is insulin or an insulin derivative.

The dose of the composition of the invention administered to an individual will vary with the particular composition, the method of administration, and the particular disease being treated. The dose is sufficient to effect a desirable response, such as a therapeutic or prophylactic response against a particular disease or condition. For example, the dosage of representative therapeutic agents (e.g., insulin) administered can be example about 0.5-1.5 IU/kg/day, 0.5-1.0 IU/kg/day, 0.6-0.9 IU/kg/day, 0.7-0.9 IU/kg/day, and 0.8 IU/kg/day.

Dosing frequency for the compositions of the invention includes, but is not limited to, at least about any of daily, twice daily, three times daily, or more frequent. The administration of the compositions of the invention can be extended over an extended period of time, such as from about a month up to about three years. For example, the dosing can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compositions described herein can be administered to an individual via various routes, including, for example, intravenous, intra-arterial, intraperitoneal, intrapulmonary, oral, inhalation, intra-vesicular, intramuscular, intra-tracheal, subcutaneous, intraocular, intrathecal, transmucosal, and transdermal. In certain embodiments, the compositions are administered by any acceptable route including, but not limited to, orally, intramuscularly, trans-dermally, and intravenously. In a preferred embodiment, the compositions are administered orally.

As discussed further herein with respect to, for example, Example 5, in simulated duodenum and jejunum intestinal fluids, there was less enzymatic or pH-induced degradation observed for compositions according to embodiments of the present disclosure. Likewise, as discussed further herein with respect to, for example, Example 6, the pharmaceutical compositions of the present disclosure are suitable to maintain stable, low levels of blood sugar after oral administration.

### EXAMPLES

### Example 1: Method for Detection and Quantification of Insulin

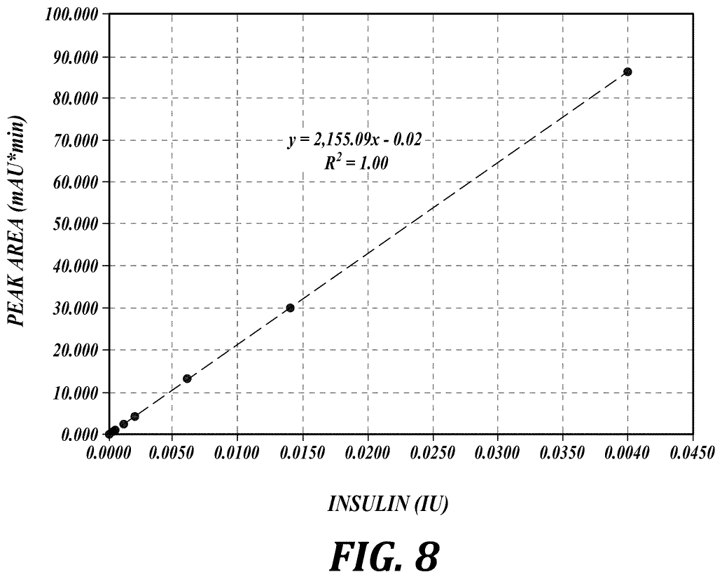

The present Example describes establishing a sensitive analytical method for detection and quantification of insulin following the US Pharmacopeia (USP) specifications. We procured insulin reference standard material from USP and identified the limit of detection and limit of quantification for supporting formulation development activities for oral gastric-resistant insulin. Exemplary high-performance liquid chromatography parameters (HPLC) are found in TABLE 1. The calibration curve is presented in FIG. 8.

TABLE 1
EXEMPLARY HIGH-PERFORMANCE LIQUID
CHROMATOGRAPHY CONDITIONS FOR INSULIN.

| Method parameters | Optimization conditions |
|---|---|
| Column | Thermo Scientific Acclaim ™ RSLC 120 C18, |
| | 2.2 μm, 120A, 2.1 × 100 mm |
| Flow rate | 0.3 mL/min |
| Wavelength | 214 nm |
| Column temperature | 30° C. |
| Injection volume | 2 μL |
| Run time | 10 min |
| Mobile phase | 0.1M potassium phosphate pH 3.1 buffer: |
| | acetonitrile:methanol (60:27:13) v/v/v |
| Elution | Isocratic |
| Needle wash | Water:acetonitrile (50:50) v/v |
| LOD (μg/mL) or IU | 3.77 or 0.00002 |
| LOQ (μg/mL) or IU | 9.44 or 0.00005 |
| Linearity calibration | 9.44-7548.97 or 0.00005-0.04000 |
| range (μg/mL or IU) | |

Abbreviations:
IU, international unit;
LOD, limit of detection;
LOQ, limit of quantification;
RSLC, rapid separation liquid chromatography.

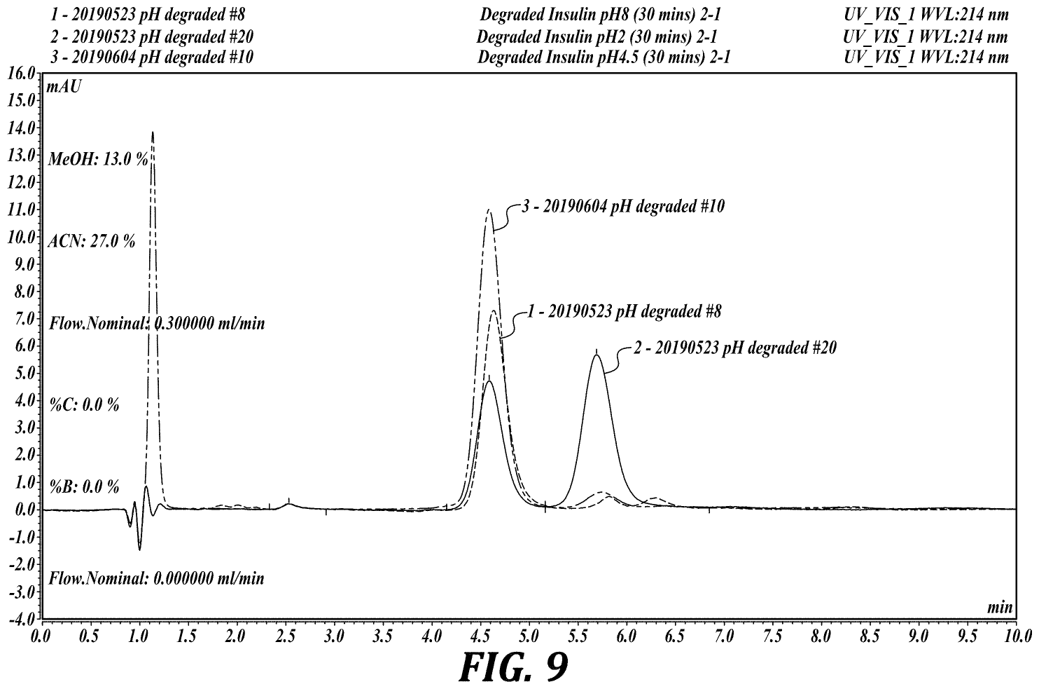

Using the established method, forced degradation studies were performed to identify the suitable pH range for insulin where less than 2% loss in insulin content was observed. The pH stability was conducted at pH 2, 4.5, and 8 where insulin was stressed for 30 minutes at 95° C. The results (FIG. 9) indicated that low pH of 2 was most detrimental to insulin stability (greater than 60% loss in content was observed using the high-performance liquid chromatography method); followed by pH 8, where less than 20% loss in insulin content was observed. Under the same conditions, at pH 4.5, insulin content was maintained at greater than 90%.

### Example 2: Formulation of Insulin

The present Example discusses formulation of thermostable, bioavailable insulin formulations according to embodiments of the present disclosure.

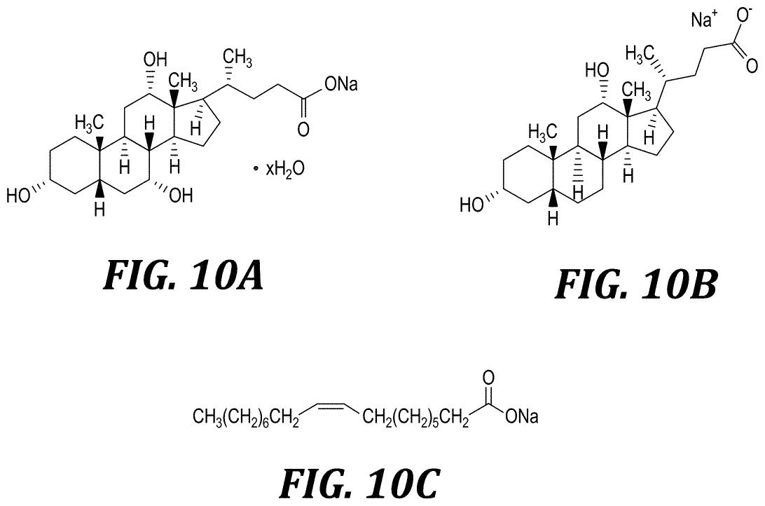

One of the key barriers to insulin absorption via the oral route is poor intestinal permeability of insulin. The present disclosure demonstrates that by forming an ion-pair with lipophilic salts such as cholic acid and fatty acids, the transport of insulin across the intestinal epithelium may be improved, mimicking the natural pathway for insulin. In the present Example, three different lipophilic salts, sodium cholate hydrate, sodium deoxycholate, and sodium oleate, were tested for their ability to form an ion-pair complex with insulin. The structures of the three lipophilic salts are shown in FIGS. 10A, 10B, and 10C, respectively.

The insulin-bile salt complex, once formed, precipitated out of aqueous solution as it was no longer water-soluble. The solution was centrifuged, and the supernatant and the pellet (complex) were tested for insulin content using high-performance liquid chromatography (HPLC). The supernatant contained no measurable insulin while the pellet (complex) contained 99% insulin used to prepare the formulation, confirming that it was complexed with bile salt.

The complexation efficiency was tested following different molar ratios of insulin to bile salt using deoxycholate where the ratio was then confirmed with other bile-salts. The results (TABLE 2) indicated that deoxycholate was most efficient in forming an ion-pair with insulin at a ratio of 1:28. Based on these results, the deoxycholate ion-pair complex was identified as the lead candidate and advanced for formulation development activities. The deoxycholate ion-pair complex is referenced elsewhere in the present disclosure as the “lead ion-pair”. The oleate insulin ion-pair complex was identified as the backup ion-pair complex candidate, and it was also advanced for formulation development activities. Even though sodium oleate had lower complexation efficiency, it was still taken forward to see whether linear fatty acid ion-pair has different insulin absorption compared to cyclic ion-pair complex with bile acid.

TABLE 2
COMPLEXATION EFFICIENCY WITH LIPOPHILIC SALTS
AND OPTIMIZATION OF THE COMPLEXATION RATIOS.

| | | | | |
|---|---|---|---|---|
| | | Motor | Complexation | CV |
| Complex name | Bile salt name | ratio | efficieney (%) | (%) |

Optimize complexation (deoxycholate)
with varied insulin to bile salt ratios

| | | | | |
|---|---|---|---|---|
| Ins-SD | Sodium | 7 | 25.1 | 4.2 |
| | deoxycholate | 10 | 37.1 | 3.3 |
| | | 28 | 98.4 | 0.5 |
| | | 140 | 99.9 | 0.1 |

Insulin complexation with bile salts

| | | | | |
|---|---|---|---|---|
| Ins-SC | Sodium cholate | 28 | 63.7 | 0.7 |
| | hydrate | | | |
| Ins-SD | Sodium | 28 | 98.4 | 0.5 |
| | deoxycholate | | | |
| Ins-SO | Sodium oleate | 28 | 90.7 | 0.5 |

Abbreviations:
CV, coefficient of variation;
Ins, insulin;
SC, sodium cholate hydrate;
SD, sodium deoxycholate;
SO, sodium oleate.

While a ratio of 1:140 was shown to have high complexation efficiency, this ratio was not selected for further study because of challenges with deoxycholate solubility under these conditions.

### Example 3: Formulation of Insulin-Bile Salt Ion-Pair as Gastric-Resistant Freeze-Dried Fast-Dissolving Tablets (FD-FDTS)

Once a preferred example lipophilic ion-pair and the corresponding ratio was identified, the ion-pair complex was formulated in gastric-resistant oral tablets. Using the ion-pair complex, several formulations were developed using resistant starch polymers, such as xanthan gum, guar gum, and pullulan and freeze-dried along with bulking agents. The compositions of the formulations are shown in TABLE 3.

TABLE 3
COMPOSITION OF FORMULATIONS CONTAINING
INSULIN-BILE SALT ION-PAIRS

| | % wt/volume | | | | | | |
|---|---|---|---|---|---|---|---|
| | Insulin- | | | | | | |
| Formulation | bile salt | Polymer | Sucrose | Mannitol | Dextran 40 | Tween 80 | Water |
| Ins-SD-FDT | 0.6 | 0.25 | 1.0 | 6.0 | 5.0 | 0.25 | 86.9 |
| (xanthan gum) | | | | | | | |
| Ins-SD-FDT | 0.6 | 0.20 | 1.0 | 6.0 | 5.0 | 0.25 | 87.0 |
| (guar gum) | | | | | | | |
| Ins-SD-FDT | 0.6 | 2.5 | 1.5 | 7.0 | 5.0 | 0.25 | 83.2 |
| (pullulan) | | | | | | | |
| Ins-SD β-CD | 0.6 | 2 | 0 | 0 | 0 | 0 | 97.4 |
| (betacyclodextrin) | | | | | | | |
| Ins-SO-FDT | 0.6 | 2.5 | 1.5 | 7.0 | 5.0 | 0.25 | 83.2 |
| (pullulan) | | | | | | | |

The formulations were freeze dried following a conservative lyophilization cycle (TABLE 5). The first tier of ion-pair formulations was developed with the lead ion pair insulin-deoxycholate.

Example processes for freeze drying and preparation of FD-FDTs will now be described with respect to certain exemplary compositions.

Step 1: Form a Complex with Insulin-Sodium Deoxycholate (SDC):

An insulin stock solution was made by dissolving 70 mg of insulin in 70 mL acetate buffer having a pH 4.0. To this insulin stock solution 78 ml of 5 mg/mL SDC was added dropwise. A colloid suspension was obtained. The suspension was stirred at 500 rpm for 30 mins in an ice bath. After stirring, the entire volume was divided into 4 different tubes and centrifuged at 2000 rpm for 15 mins. The precipitate complex was collected. The supernatant was collected and tested for the remaining insulin using HPLC to test for the complexation efficiency (calculated as 99%).

Step 2: Encapsulate the Complex with 5% Hydroxypropyl-Beta Cyclodextrin (BETA HPCD):

Firstly, 30 mL of beta HPCD was made by dissolving 1.5 g of beta HPCD in 30 mL (5 wt/vol %) of DI water. Then, 15.5 mL of the beta HPCD solution was added to the precipitate complex from Step 1, followed by stirring for 2 hours at room temperature. This is Formulation B.

Preparation of Formulations C and D:

For preparation of Formulations C and D, additional 2% sucrose, 3% Mannitol (Formulation C) or 2% sucrose, 3% Mannitol 0.05% Tween 80 (PS-80) (Formulation D) were added to the beta HPCD/insulin complex solution under Step 2 for encapsulation with 5% beta HPCD.

A summary of example Formulations according to embodiments of the present disclosure is provided in TABLE 4.

TABLE 4
COMPOSITIONS OF FORMULATIONS (% W/W) IN DRY DOSAGE FORM

| | Insulin | SDC | HPCD | Sucrose | Mannitol | Dextran 40 | Tween 80 |
|---|---|---|---|---|---|---|---|
| Formulation | (%) | (%) | (%) | (%) | (%) | (%) | (%) |
| A | 9.09 | 50.5 | 40.41 | x | x | x | x |
| B | 5.66 | 31.43 | 62.91 | x | x | x | x |
| C | 3.48 | 19.3 | 38.63 | 15.44 | 23.16 | x | x |
| D | 3.41 | 18.91 | 37.85 | 15.12 | 22.69 | x | 2.02 |
| E | 4.52 | 24.89 | 50.27 | x | x | 20.32 | x |
| F | 6.48 | 35.98 | 28.77 | x | x | 28.77 | x |
| G | 3.77 | 20.91 | 16.74 | 16.74 | 25.1 | 16.74 | x |
| H | 3.02 | 16.72 | 33.46 | 13.37 | 20.06 | 13.37 | x |
| I | 5.48 | 30.41 | 60.88 | x | x | x | 3.23 |

Preparation of Freeze-Dried Tablets:

The encapsulated complex mixture was further diluted to 70 mL with DI water. The resulting mixture was loaded into blisters and a 2 mL vial. Each vial and blister were filled with 0.5 mL of the mixture. A total of 70 blisters and 70 vials were filled. Both blister sheets and vials were freeze dried following the established freeze-drying cycle. This resulted in a product (vial or tablet) containing ˜0.5 mg or ˜13.25 IU insulin after the completion of the freeze-drying process.

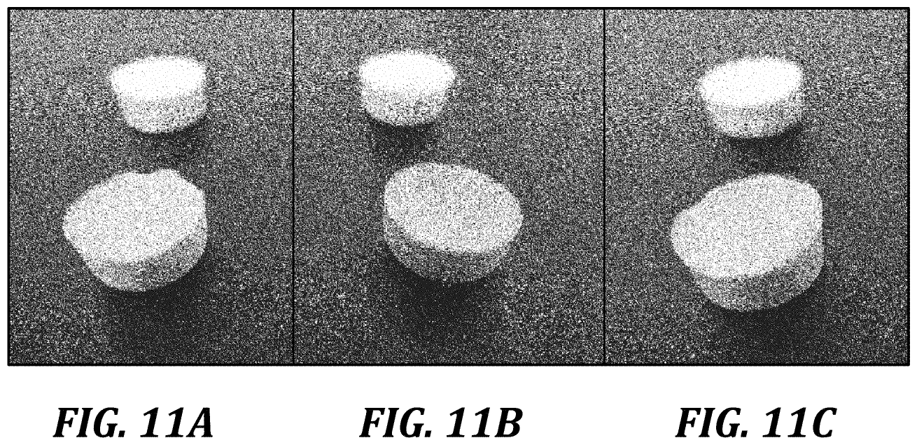

Once the formulation was down selected based on insulin content recovery, the same composition was used in formulating the backup ion-pair insulin-oleate. The images of the resulting FD-FDTs are shown in FIGS. 11A-11C.

TABLE 5
FREEZE DRY CYCLE CONDITIONS FOR PRODUCTION
OF INSULIN CONTAINING FDTS.

| | 1 | 2 | 3 | 4 | 5 | 6 | 7 | 8 |
|---|---|---|---|---|---|---|---|---|
| Stage 1 - Freeze | | | | | | | | |
| Temperature (° C.) | 4 | 4 | −45 | −45 | −10 | −10 | −45 | −45 |
| Time (min) | 0 | 180 | 180 | 180 | 120 | 180 | 120 | 180 |
| Vacuum (mtorr) | 100 | 100 | 100 | 100 | 100 | 100 | 100 | 100 |
| Stage 2 - Primary drying | | | | | | | | |
| Temperature (° C.) | −25 | −25 | 4 | 4 | — | — | — | — |
| Time (min) | 60 | 1440 | 120 | 360 | — | — | — | — |
| Vacuum (mtorr) | 100 | 100 | 100 | 100 | — | — | — | — |
| Stage 3 - Secondary drying | | | | | | | | |
| Temperature (° C.) | 4 | — | — | — | — | — | — | — |
| Time (min) | 60 | — | — | — | — | — | — | — |
| Vacuum (mtorr) | 100 | — | — | — | — | — | — | — |

The tablets were evaluated based on physical appearance, residue, and insulin content/potency (TABLE 6). Insulin content recovery was lowest from the xanthan gum formulation and this formulation was eliminated from further evaluation.

While the FDT formulation containing guar gum did show complete recovery of insulin content post freeze-drying, the tablet physical appearance was not optimal, and surface and edge cracks were observed. In view of the combined results based on insulin content recovery and tablet physicochemical appearance, the FDT formulation containing pullulan was identified as the lead gastric-resistant FDT formulation for both ion-pair.

TABLE 6
INSULIN CONTENT RECOVERY FROM FD-FDTS.

| | Insulin content | |
|---|---|---|
| | Drug recovery from | CV |
| Formulation | FD-FDTs (%) | (%) |
| Ins-SD-FDT (xanthan gum) | 21.0 | 4.0 |
| Ins-SD-FDT (guar gum) | 99.5 | 0.8 |
| Ins-SD-FDT (pullulan) | 100.7 | 0.1 |
| Ins-SD β-CD (beta-cyclodextrin) | 100.0 | 0.9 |
| Ins-SO-FDT (pullulan) | 100.9 | 0.5 |

### Example 4: Thermostability of Insulin Formulation

The present Example discusses thermostability of pharmaceutical compositions according to embodiments of the present disclosure that include insulin.

Temperature is another barrier to uptake of insulin, as improper storage of insulin has been shown to decrease its potency. To test the thermostable potential of the developed oral insulin FDTs, we staged a three-month stability study where the FDTs containing insulin were stored under different temperature conditions along with unformulated insulin (liquid) as a control. Duplicate samples were tested at initial, week 2, week 4, week 8 and week 12 time points to determine insulin content using the analytical HPLC method established previously in Example 1 (see FIGS. 3A-3C for results).

Freeze-dried tablets as described in Example 3 and unformulated insulin control were stored at 2-8° C., 30° C. and 65% relative humidity (RH), and 40° C. and 75% RH, and tested for insulin content at week 0, week 2, week 4, week 8, and week 12 following the US Pharmacopeia (USP) specifications for insulin to establish a sensitive analytical high-performance liquid chromatography (HPLC) method for detection and quantification of insulin.

Lyophilized insulin was dissolved in a vial or 2 lyophilized tablets using 1 mL of an acetate buffer. The dissolved insulin was vortexed until fully disintegrated and placed in an ice before use. The appearance of the dissolved insulin was similar to pre-lyophilized sample. 8 mL of 1% acetic acid and methanol and were gently turned upside down several times to mix the solutions and then placed in ice or 4° C. for 1 hour. The resulting cloudy mixture was centrifuged at 3500 rpm for 20 mins to separate the excipients at the bottom. The supernatant was transferred to LC vial, where one sample was tested as-is and one sample was diluted 10 times.

The soluble insulin was tested on high-performance liquid chromatography (HPLC) (Thermo Scientific Dionex UltiMate™ 3000 series) and UV-Vis detector The extracted insulin (injection volume 2 μL) was analyzed using an Acclaim™ RSLC 120 C18 column (2.2 μm, 2.1×100 mm; part #068982; Thermo Fisher Scientific, Waltham, Mass., USA) at 30° C., at flow rate 0.3 mL/minute, using an isocratic method with a mobile phase 13% methanol/27% acetonitrile/60% 0.1M potassium phosphate pH 3.1. The retention time of insulin was approximately 4.6 minutes. Empower chromatography software (Waters Corporation) was used to automatically integrate HPLC chromatograms.

The percent insulin remaining in the tablets stored under these conditions are illustrated in FIGS. 3A-3C, where FIG. 3A graphically illustrates thermal stability of control insulin and freeze-dried oral insulin tablets according to embodiments of the present disclosure at 2° C.-8° C.; FIG. 3B graphically illustrates thermal stability of control insulin and freeze-dried oral insulin tablets according to embodiments of the present disclosure at 30° C. and 65% relative humidity (RH); and FIG. 3C graphically illustrates thermal stability of control insulin and an oral insulin tablet according to an embodiment of the present disclosure at 40° C. and 75% RH. As shown, the oral insulin tablets according to embodiments of the present disclosure have far greater thermostability than the control insulin.

The results of the present Example show that insulin-sodium deoxycholate (Ins-SD) ion pair protected by polymers such as beta-cyclodextrin or pullulan and freeze dried as solid dosage forms offered protection to insulin from temperature and humidity induced degradation at both 30° C./65% relative humidity (RH) and 40° C./75% RH storage conditions. Under these conditions, insulin was stable for up to 12 weeks at 30° C./65% RH and up to 4 weeks at 40° C./75% RH, with none to minimal loss in potency. Liquid unformulated insulin (control), lost 20% and 50% insulin content at the end of 2 weeks at 30° C./65% RH and 40° C./75% RH, respectively.

Based at least upon the results described herein, it is believed that HP-β-CD polymer, used in preparing the formulations described in the present Example, encapsulates and stabilizes the lipophilic insulin ion-pair to form a heat-stable pharmaceutical composition. In this regard, it is believed that encapsulation within HP-β-CD protects insulin from temperature-induced degradation, which is in contrast to unformulated insulin (control), which lost >50% insulin content within 2 weeks under similar storage conditions.

### Example 5: Characterization of the Developed FD-FDTS for Physicochemical Properties and Insulin Content and Stability

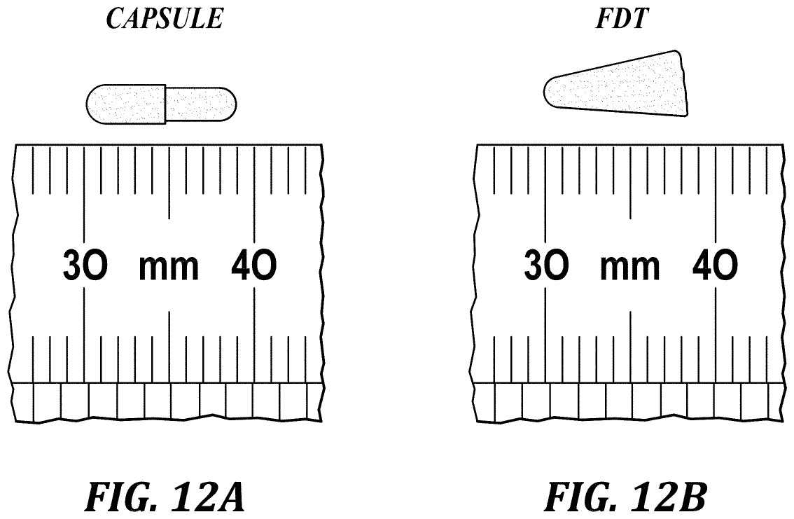

With oral administration, the first barrier for insulin delivery is encountered in the stomach, where insulin is degraded by pH and proteolytic enzymes. Using the lead FD-FDT formulation identified in Example 3 above, we conducted in vitro evaluation using the static gastrointestinal use testing (GUT) model. The purpose of this evaluation was to test whether the bile-insulin ion-pair encapsulated within the resistant starch polymer is protected from pH and enzymatic degradation encountered in the gastrointestinal compartments. Since the in vivo feasibility of insulin FDTs is proposed in a diabetic rodent model, we scaled down the size of the FDTs based on limited size of the oral cavity in rats. We also included commercially available gelatin oral capsules fit for oral delivery to rats (FIGS. 12A and 12B). Biorelevant dissolution media simulating the pH and enzymatic conditions in the different sections of gastrointestinal tract, such as fasted state simulated gastric fluid (FASSFG), and intestinal fluid in the duodenum and jejunum (TABLE 7) were generated following the composition recipe reported by Jantratid et al. (2009).

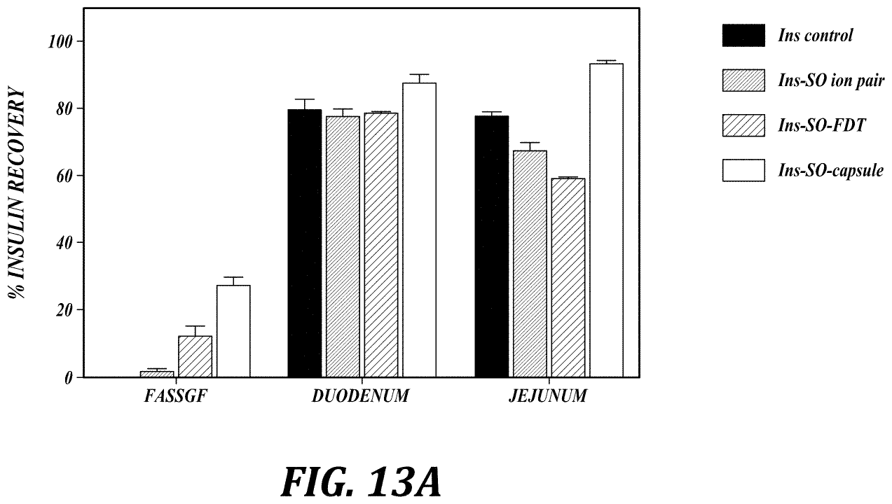

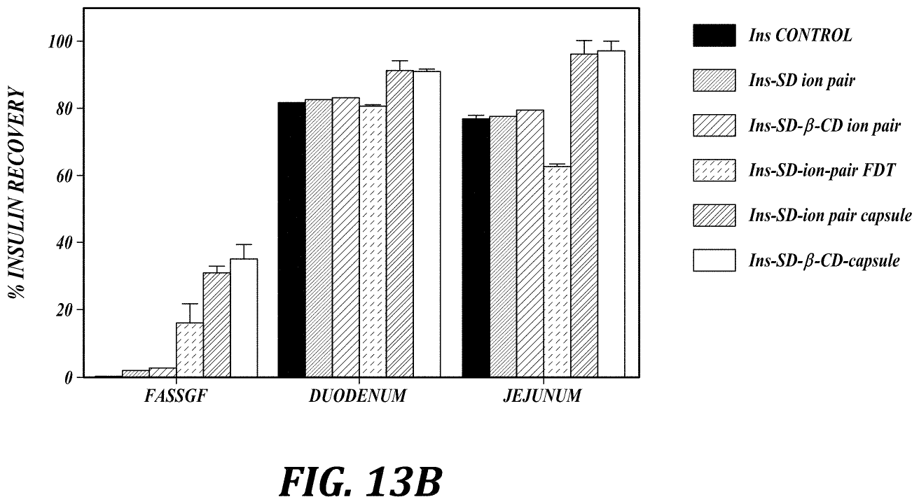

Samples were incubated in the different fluid media maintained at 37° C. and at the 30-minute time point, removed and tested for insulin content. Unformulated insulin was used as a control for comparative evaluation (FIGS. 13A and 13B).

TABLE 7
COMPOSITION OF DISSOLUTION MEDIA
USED IN INVITRO GUT MODEL.

| Composition | |
|---|---|
| (mM) | Concentration |
| Fasted state simulated gastric fluid | |
| (FASSGF) | |
| Sodium taurocholate | 80 μM |
| Lecithin | 20 μM |
| Pepsin | 2,000 U/mL |
| Sodium chloride | 34.2 mM |
| Pepsin | 0.1 mg/mL |
| Hydrochloric acid, q.s. | — |
| pH | 1.57 |

Simulated intestinal fluid: Duodenum

| | |
|---|---|
| Sodium taurocholate | 10 |
| Lecithin | 2 |
| Glyceryl monooleate | 5 |
| Sodium oleate | 0.8 |
| Maleic acid | 55.02 |
| Sodium hydroxide | 81.65 |
| Sodium chloride | 125.5 |
| Porcine pancreas | 0.05 mg/mL |
| pH | 6.00 |

Simulated intestinal fluid: Jejunum

| | |
|---|---|
| Sodium taurocholate | 10 |
| Lecithin | 2 |
| Glyceryl monooleate | 5 |
| Sodium oleate | 0.8 |
| Maleic acid | 55.02 |
| Sodium hydroxide | 81.65 |
| Sodium chloride | 125.5 |
| Porcine pancreas | 0.05 mg/mL |
| pH | 6.8 |

The results from the in vitro evaluation indicated that while insulin alone and insulin-ion pair complex degraded almost completely in the presence of gastric fluid, the insulin ion-pair formulated as a FDT or protected by gelatin capsules were able to protect insulin from stomach acid degradation maintaining insulin content greater than 30% indicating the importance of protecting insulin from degradation induced by stomach acid and enzymes. Comparing acid and enzymatic protection offered by gelatin capsule versus FDT, it appeared that insulin ion-pair was better protected within capsule than within FDT. In simulated duodenum and jejunum intestinal fluids, there was less enzymatic or pH-induced degradation observed for all the test groups.

### Example 6: In Vivo Testing of Insulin Formulation

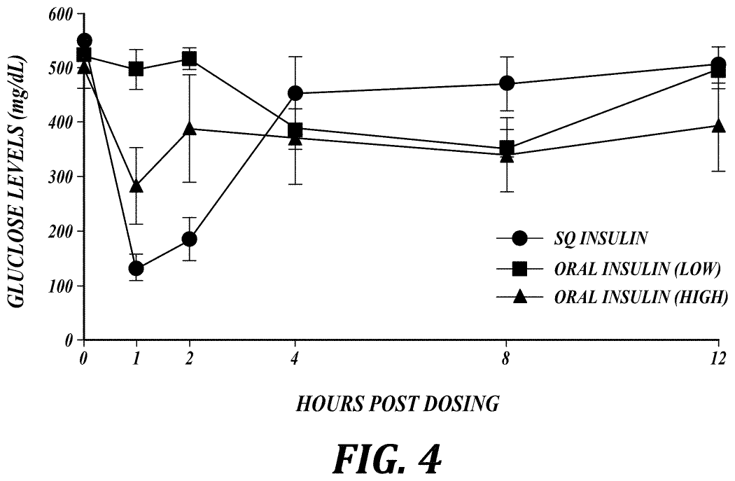

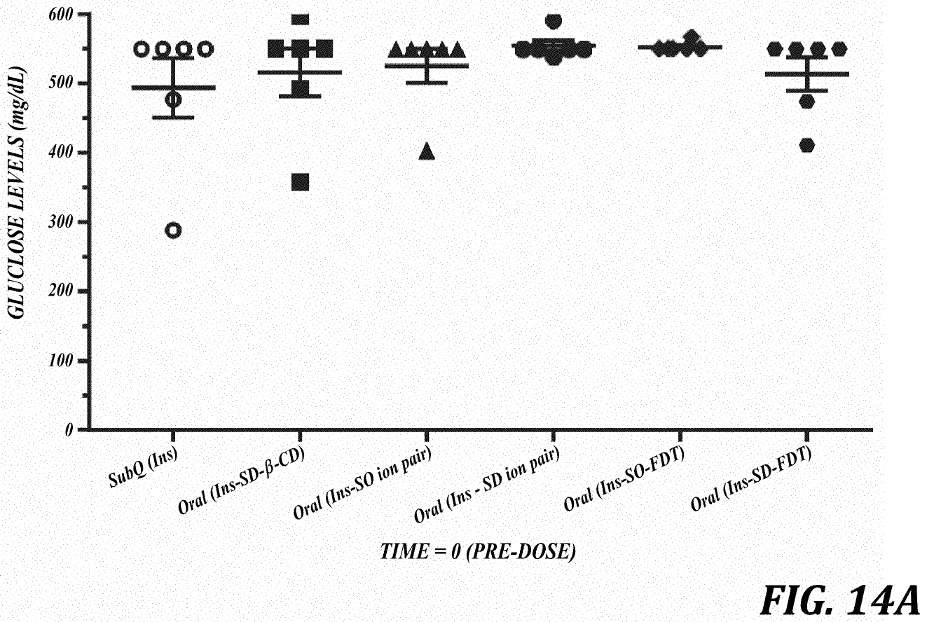

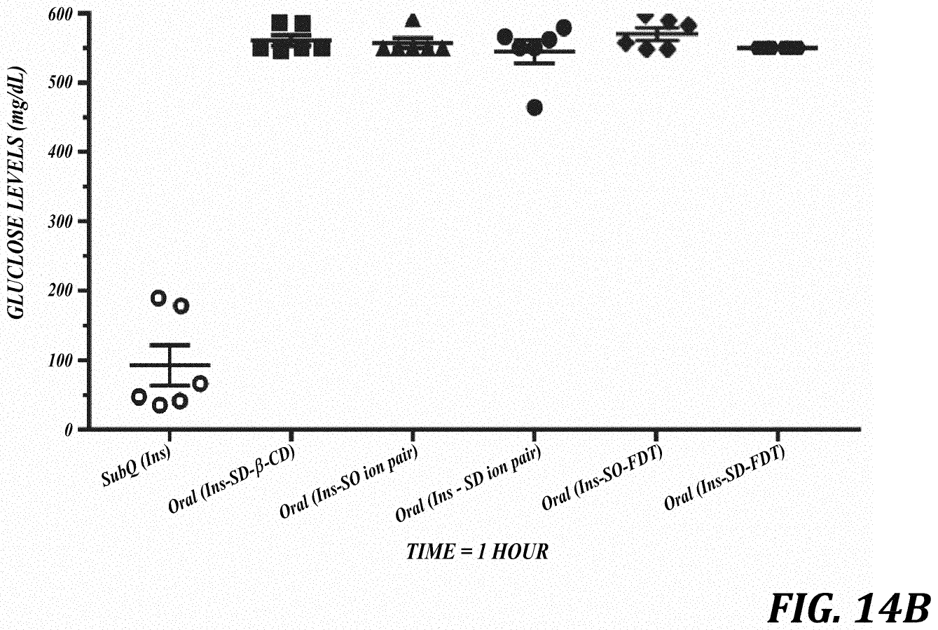

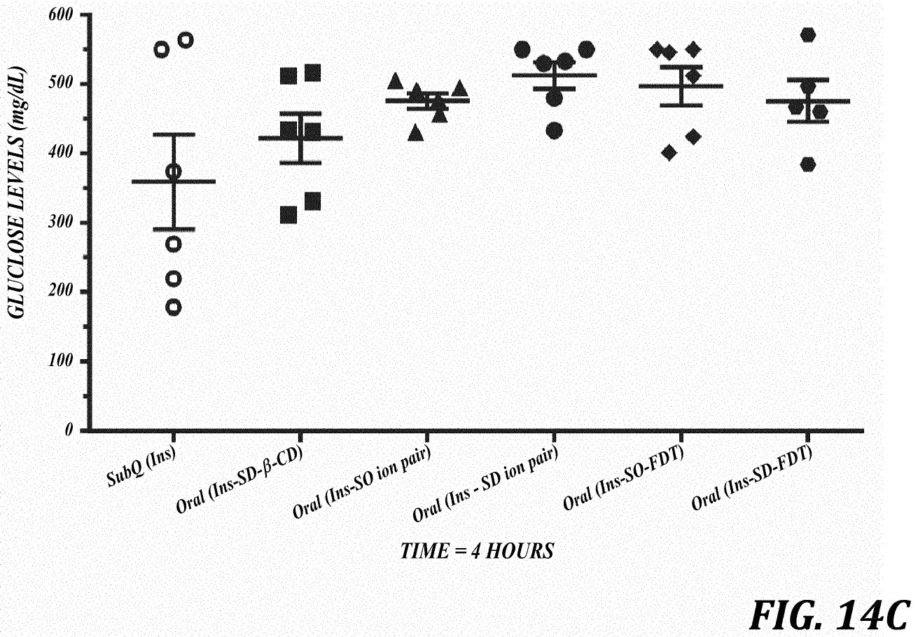

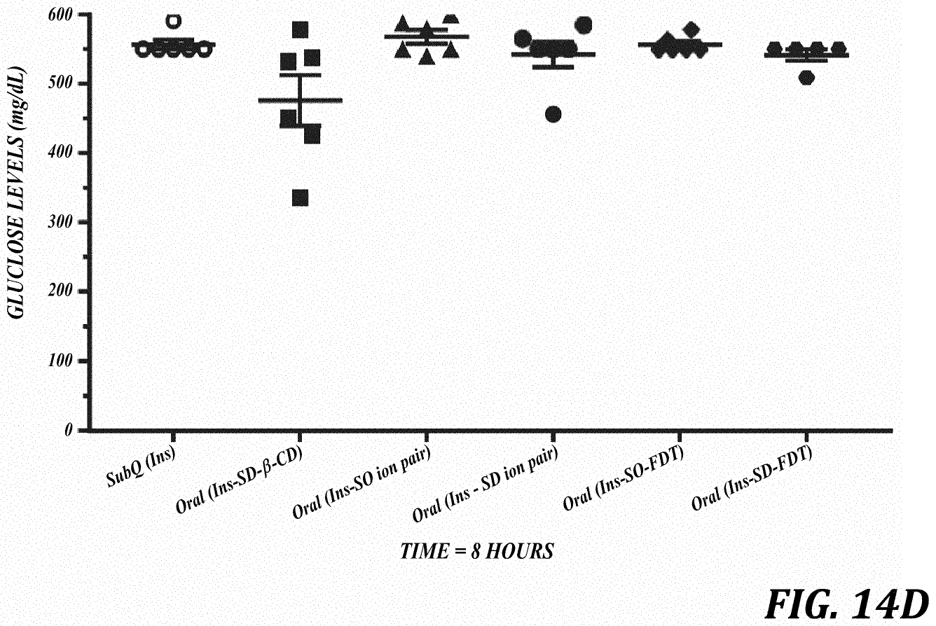

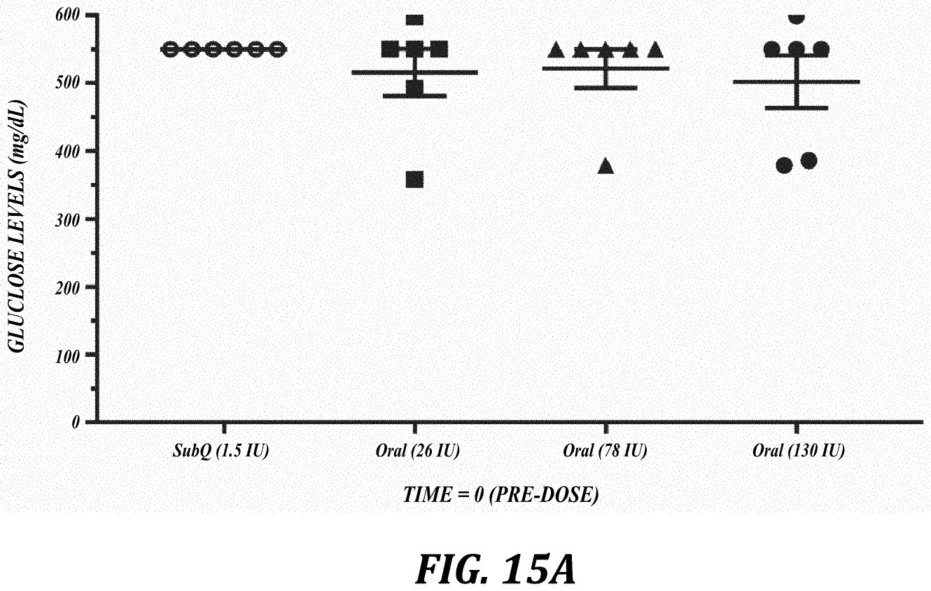

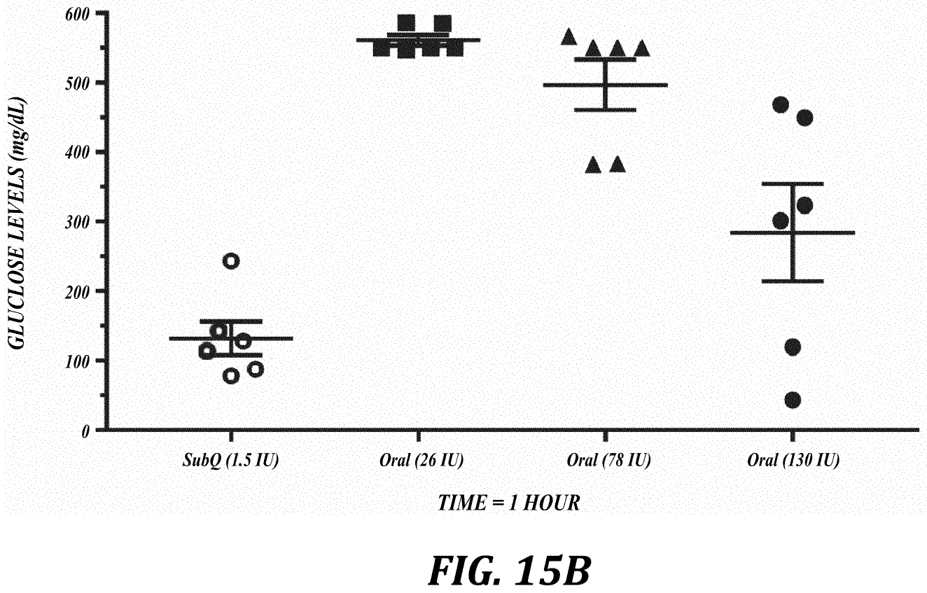

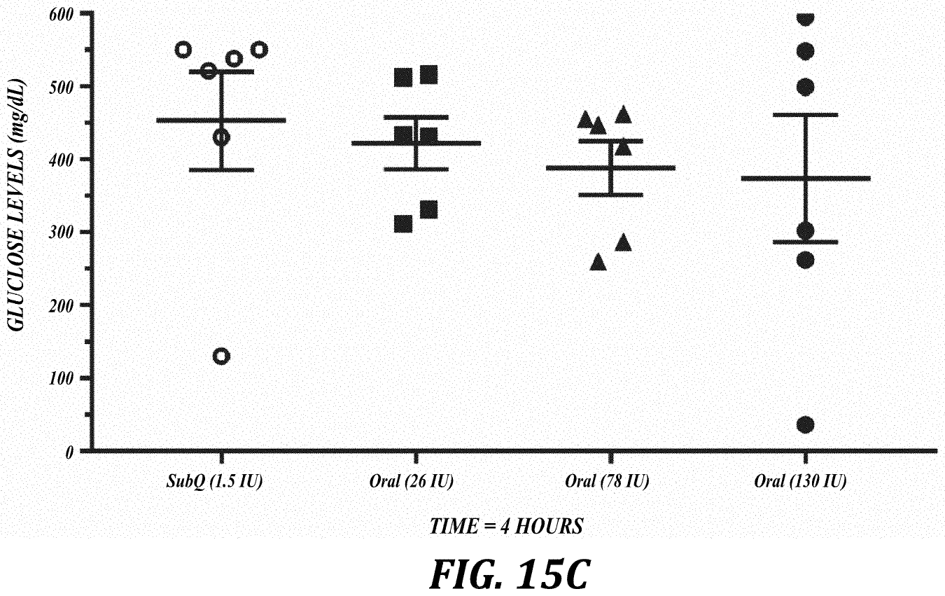

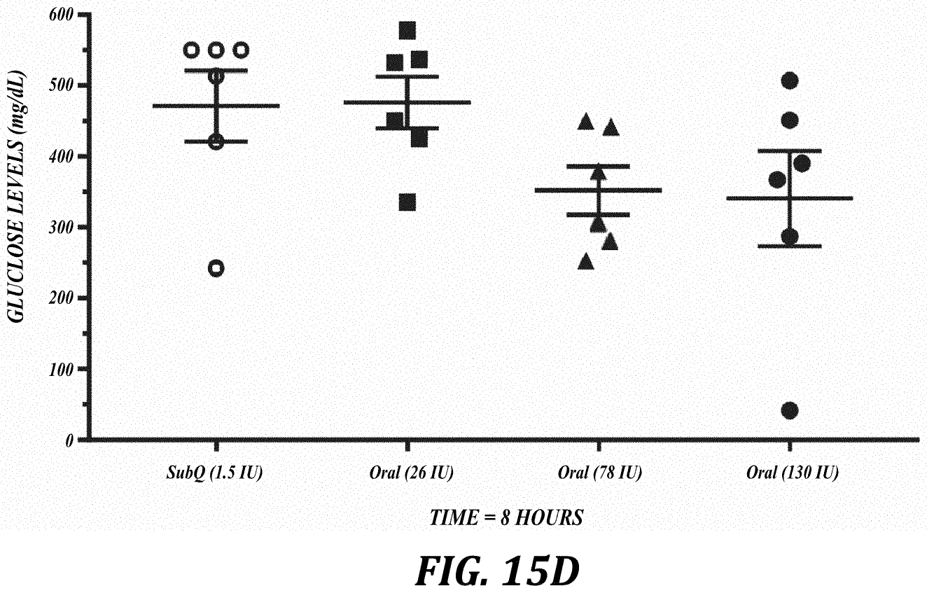

The present Example describes a) selection of the dosage presentation for the lead ion-pair and the dose (see TABLE 8, FIGS. 4 and 14A-14D) and b) validation of the lead ion-pair formulation in their ability to lower glucose levels upon oral administration of the formulations (see FIGS. 15A-15D). In part a) of the present Example, we compared the capsule and FDT presentation formats for both ion-pair formulations. We also included β-CD-encapsulated ion-pair complex to see whether the encapsulation of ion-pair within β-CD offers further improvement in absorption of insulin. Liquid insulin given subcutaneously (subQ) was used a comparator in both studies.

TABLE 8
PILOT STUDY GROUPS AND DOSING SCHEDULE.

| | Number of | | | | Blood |
|---|---|---|---|---|---|
| | animals | | | | collection |
| | (Male + | Group | Route (dosage | | time |
| Group | Female) | designation | presentation) | Dose | points |
| 1 | 3M + 3F | Insulin | Subcutaneous | 5 IU/kg | 0 min, |
| | | control | | (1.5 | 30 min, |
| | | | | IU/rat) | 60 min, |
| 2 | 3M + 3F | β-CD- | Oral (capsule) | 87 IU/kg | 90 min, |
| | | encapsulated | | (26 | 2 h, |
| | | ion-pair | | IU/rat) | 4 h, 8 h |
| | | formulation 1 | | | |
| 3 | 3M + 3F | Ion-pair | Oral (capsule) | 87 IU/kg | |
| | | formulation 1 | | (26 | |
| | | | | IU/rat) | |
| 4 | 3M + 3F | Ion-pair | Oral (capsule) | 87 IU/kg | |
| | | formulation 2 | | (26 | |
| | | | | IU/rat) | |
| 5 | 3M + 3F | Ion-pair | Oral (FDT) | 87 IU/kg | |
| | | formulation 1 | | (26 | |
| | | | | IU/rat) | |
| 6 | 3M + 3F | Ion-pair | Oral (FDT) | 87 IU/kg | |
| | | formulation 2 | | (26 | |
| | | | | IU/rat) | |

Results of portion a) of the present Example indicate that none of the test groups formulated as FDT or capsule lower the glucose levels within the first hour of administration while the subQ route lowered the glucose levels by about 80% (from 600 mg/dL to 100 mg/dL) in the same time frame. At T=4 hours, the β-CD-encapsulated insulin-SD ion-pair showed 30% drop (from 600 mg/dL to ˜400 mg/dL) in glucose level and maintained these lowered glucose levels at T=8 hours, suggesting the possibility of slow sustained release of insulin from the β CD-encapsulated ion-pair. The subQ group retained glucose levels at 600 mg/dL, similar to what was obtained at T=0. Based on this outcome, the β-CD-encapsulated insulin-SD ion pair was advanced to the confirmation study (TABLE 9, FIGS. 15A-15D) for testing effect additional doses on glucose levels in rats.

TABLE 9
CONFIRMATION STUDY GROUPS AND DOSING SCHEDULE.

| | Number of | | | | Blood |
|---|---|---|---|---|---|
| | animals | | Route | | collection |
| | (Male + | Group | (dosage | | time |
| Group | Female) | designation | presentation) | Dose | points |
| 1 | 3M + 3F | Insulin | Sub- | 5 IU/kg | 0 min, 1 h, |
| | | control | cutaneous | (1.5 IU/rat) | 2 h, 4 h, |
| 2 | 4M + 2F | β-CD | Oral | 223 IU/kg | 8 h, 12 h, |
| | | ion-pair | (capsule) | | 24 h |
| | | formulation | | (78 IU/rat) | |
| | | (medium | | | |
| | | dose) | | | |
| 3 | 3M + 3F | β-CD | Oral | 371 IU/kg | |
| | | ion pair | (capsule) | | |
| | | formulation | | (130 IU/rat) | |
| | | (high dose) | | | |

The results from portion b) of the present Example validate the slow sustained-release effect of β-CD-encapsulated insulin-ion pair in lowering and maintaining glucose levels. As seen from the data, the subQ group showed the highest drop in glucose levels (˜80%) at T=1 hour (600 mg/dL to ˜100 mg/dL). The FDT high dose test group showed an ˜60% drop in glucose levels (500 mg/dL to 300 mg/dL) and maintained those levels for up to 8 hours. This noticeable result validates the sustained effect of the β-CD encapsulated insulin ion-pair formulation where the glucose levels were maintained through the 8-hour timepoint, unlike the subQ administered insulin where the glucose levels rose back to −500 mg/dL in the same time frame.

These results indicate that oral heat stable insulin, i.e., the pharmaceutical compositions of the present disclosure, have been developed into a sustained release product capable of providing a steady low level of insulin for maintaining blood glucose levels. The developed pharmaceutical compositions demonstrated robustness and superior thermostability compared to liquid insulin control, retaining potency without refrigeration, and making it suitable for low- and middle-income countries where prevalence of diabetes is high and rising. Additionally, the ease-of-use of our freeze-dried oral insulin tablet can improve the management of diabetes for special populations including children and people with swallowing difficulties. Since the methods of the present disclosure use, in certain embodiments, freeze drying or lyophilization, the resulting pharmaceutical compositions have a highly porous matrix, which is softer compared to conventional compressed tablets, which are hard. Additionally, the methods of the present disclosure allow for flexibility in providing product/tablets in different sizes and shapes, which may be easier to swallow than conventional tablets.

Furthermore, the heat-stable tablets are produced using a freeze-drying manufacturing platform, enabling a rapid development timeline and potential to reduce the costs of insulin distribution.

### Example 7: In Vitro Tissue Evaluation

In the present Example, the 4 formulations (Formulation A (unformulated insulin) and Formulations B-D (formulations described further herein with respect to Example 3) were tested using in vitro intestinal tissues (Epilntestinal™) to evaluate the effect of 4 oral insulin formulations on Barrier Function, inflammation, and the ability to deliver insulin levels when added to the Epilntestinal™ in vitro tissue model.

The purpose of the in vitro tissue is to evaluate the effect of 4 insulin Formulations A, B, C, and D (see TABLE 4) on Barrier Function, Inflammation and the ability to deliver insulin levels when added to the Epilntestinal™ (SMI-100) in vitro tissue model. Briefly, the tissues (in inserts) are placed into each well (hanging top) of a 12-well plate containing 5.0 mL of pre-warmed (to 37°±1° C.) medium (SMI-100-MM). The tissues are equilibrated by pipetting 100 uL of the pre-warmed media onto the apical surface of the tissue at standard culture conditions (SCC, 37°±1° C. and a humidified atmosphere of 5% CO2±1%) overnight. Following overnight incubation, SMI-100 tissues are transferred into a 24-well plate containing transepithelial electrical resistance (TEER) buffer and basal (0 min) TEER is measured using an epithelial volt-ohm meter EVOM® and the EndOhm-12 chamber (World Precision, Sarasota, Fla.), following the procedure described in the protocol described further herein with respect to the present Example. For testing the effect of Formulations on tissue integrity and inflammation, gene expression analysis is conducted using qPCR method. Total RNA is isolated (from the tissue lysates stored at −80° C.) using RNAqueous kit from Invitrogen following manufacturer's procedure. Total RNA is used to synthesize cDNA with Qiagen reagents following manufacturer's protocol. qPCR is performed using RT-PCR panel for inflammation. Gene expression of (IL-6, TNF-a, IL-2, IL10, myeloperoxidase and RPLP0) is determined to estimate the level of tissue inflammation (potential risk for toxicity) due to exposure to the formulations

The results from the barrier function testing show a drop in TEER >90% for the tissues treated with insulin Formulations A-D compared to the untreated tissues control group (Negative Control NC), indicating significant drop in tissue membrane resistance and potential increase in tissue permeability. The results from the inflammation testing-based gene expression analysis of key inflammatory marker genes (IL-2, IL-6, IL-10, MPO, TNF) show that in the tissues treated with the Formulations the gene expression is upregulated by ˜10% for, while for the house keeping gene (RPLP0) the levels were increase by ˜30%. Although the levels are slightly higher than the untreated control, it is not known whether the levels are reversible and return to normal upon removal of the treatment.

Preparation of Epiintestinal (SMI-100) Tissues

SMI-100 tissues are removed from packaging. Each insert is placed into each well (hanging top) of a 12-well plate containing 5.0 mL of pre-warmed (to 37°±1° C.) medium (SMI-100-MM). 100 uL of the pre-warmed media is pipetted onto the apical surface of the tissue. At standard culture conditions (SCC, 37°±1° C. and a humidified atmosphere of 5% CO2±1%) the solution is equilibrated overnight.

Exposure of Epiintestinal Tissues

Following overnight incubation, SMI-100 tissues are transferred into a 24-well plate containing TEER buffer and at 0 min basal TEER is measured using an epithelial volt-ohm meter EVOM® and the EndOhm-12 chamber (World Precision, Sarasota, Fla.), following the procedure outlined in Section 8.4 below. Tissues are transferred to each well of a 24-well plate containing pre-warmed 0.5 mL of receiver buffer (1.98 g/L glucose in 10 mM HEPES, lx Hank's Balanced (Salt Solution) pH 7.4) and equilibrated for 15 min in the incubator at standard culture conditions (SCC, 37°±1° C. and a humidified atmosphere of 5% CO₂±1%). Tissues are moved to a new 24-well plate (30 min plate) with 0.5 mL of receiver buffer and dose n=6 tissues topically with 200 μL of each test article (donor solutions). Untreated tissues serve as negative control (NC). After 30 minutes of elapsed time, the tissues are moved to a new well of the 24-well plate, (designated 1 hr) with 0.5 mL of receiver buffer. After 1 hr of elapsed time, the tissues are moved to a new well of the 24-well plate (designated 1.5 hr) with 0.5 mL of receiver buffer. After 1.5 hr of elapsed time, the tissues are moved to a new well of the 24-well plate (designated 2 hr) with 0.5 mL of receiver buffer. At the end of the 2 hr time point, donor solutions and receiver solutions are collected into pre-labeled airtight tubes and store at 4° C. until ready to process for insulin ELISA. The tissues are rinsed with TEER buffer and measure TEER (2 hr) using an epithelial volt-ohm meter EVOM® and the EndOhm-12 chamber (World Precision, Sarasota, Fla.), following the procedure outlined herein below. Lyse n=3 tissues in PBS from each treatment condition. the lysates are saved for insulin ELISA. Lyse n=3 tissues in RNA lysis buffer and store at −80° C. until further processing for RNA isolation.

Barrier Integrity—Measurement of TEER

The EVOM2 is calibrated one time on the day of measurement prior to measurement of TEER of Epilntestinal™ tissues. The ENDOHM-12 is filled with 100 mM KCl just below the rim and is placed the respective cover on top, and is equilibrated for 20 minutes at room temperature with the lid on the chamber. The EVOM2 Power knob is turned to ON. The Zero mV (voltage) is adjusted with adjustment screw and a small flathead screwdriver until the display read 0.0. Liquid is removed from the EndOhm chamber and is replaced with 2.5 mL of TEER buffer. The Blank CaliCell insert (CaliCell-12, World Precision Instruments) is inserted and the lid is placed back on the chamber, ensuring that the buffer inside the insert is not in contact with the buffer in the bottom of the Endohm chamber. The mV value displayed is recorded. The Mode knob is turned to Ohms, the value displayed (example reading=15 S2) is recorded, and then the Mode knob is turned back to mV before removing the lid. This is the BACKGROUND RESISTANCE. The CaliCell-12 insert is inserted and the level of the TEER buffer is drawn down to the top of the insert. The lid is placed back on the chamber. The mV value displayed is recorded. The Mode knob is turned to Ohms, the value displayed (example reading=425 Ω) is recorded, and then the Mode knob is turned back to mV before removing the lid. This is the TOTAL RESISTANCE. The MEMBRANE RESISTANCE (TOTAL RESISTANCE-BACKGROUND RESISTANCE) is calculated. Calculated MEMBRANE RESISTANCE should be ±10% of the calibrated value on the CoA provided with the CaliCell. Using a blank insert (MILCEL-MTK) the background resistance is measured and should be about 5-20 Ω/cm². The tissue inserts are transferred to a 6-well plate containing small amount of KCl and the top surfaces are gently rinsed the top surface of the tissue by adding 0.5 mL KCl and are aspirated. A 24-well plate is prepared with 0.25 mL of sterile KCl (100 mM) per well. Each individual tissue is transferred into its own well of the 24-well plate containing 0.25 mL of KCl/well. 400 μL of sterile KCl is added to the apical side of each tissue. The insert is transferred into the EndOhm chamber and is centered. The cap is placed onto the chamber, ensuring that the top electrode is making contact with the sterile KCl while checking for any air bubbles. The resistance (mode knob set to R) is recorded and then the PD reading (mode knob to V) is recorded. The tissue insert is removed from the chamber and is placed back into the 24-well plate containing KCl. These steps are for all tissues.

After the TEER measurements are taken, the KCl is removed from all of the wells and tissue culture inserts. The inserts are placed back into their corresponding wells in the receiver buffer (after the 0 min TEER measurement) and the tissues are lysed (at the end of 2 hr TEER measurement).

Insulin ELISA

Insulin levels are measured (from donor solution, receiver solution, tissue lysates) using Insulin ELISA kit (abcam—Cat #ab278123) and following manufacturer's procedure.

Gene Expression Analysis

Total RNA is isolated (from the lysates stored at −80° C.) using RNAqueous™ kit from Invitrogen following manufacturer's procedure. Total RNA is used to synthesize cDNA with Qiagen reagents following manufacturer's protocol. qPCR is performed using RT-PCR panel for inflammation. Gene expression of (IL-6, TNF-a, IL-2, IL10, myeloperoxidase and RPLP0) is determined.

### Example 8: In Vivo Testing of Pharmaceutical Compositions

The present Example demonstrates the effect the effect of orally delivered a pharmaceutical formulation including an insulin-ion-pair encapsulated in 2% beta-HP-CD versus a formulation insulin-ion-pair encapsulated in 5% beta-HP-CD on glucose lowering effects in a diabetic rodent model.

Briefly, the male Sprague Dawley rodents (200-250 grams) are fasted 4 hours before given 65 mg/kg intraperitoneal (IP) streptozotocin (STZ) to induce diabetes and randomized into treatment groups based on the post-STZ (˜1 week) baseline glucose levels (see TABLE 10). Only rodents with hyperglycemia (glycose levels >200 mg/ml) are used in the study. Rodents are housed on a 12-hour light/dark cycle with no more than 2 rats per cage depending on size and acclimatized for not less than 5 days. Blood glucose levels are measured using calibrated Glucocard Vital glycometers (Arkray, Minneapolis, Minn.) and levels are reported as mg/dL. For each timepoint, blood (<5 uL) is acquired from a tail snip and directly applied to a glucose test strip. Data for each quantitative timepoint is presented as the average+/−SEM (standard error of the mean). Differences between vehicle control and each treatment groups are determined by Student's t-test using GraphPad Prism (current version) with an alpha threshold of 0.05.

TABLE 10
IN VIVO TREATMENT DETAILS

| GROUP | | GROUP | DAYS OF | | |
|---|---|---|---|---|---|
| # | TREATMENT | SIZE | DOSING | DOSE | ROUTE |
| 1 | Formulation | 15 | Once | 100 IU | Oral |
| | (2% beta-HP-CD) | | | | |
| 2 | Formulation | | | 60 IU | Oral |
| | (5% beta-HP-CD) | | | | |
| 3 | Formulation | | | 104 IU/vial | Oral |
| | (5% beta-HP-CD) | | | | |
| 4 | Insulin | | | 26 IU/mL | SC |
| | (SC control) | | | | |

Insulin and blood glucose levels were measured before administration, at 30 minutes, 60 minutes, 90 minutes, 2 hours, 4 hours, 8 hours, and 24 hours after dosing.

Additionally, bodyweight was measure before and after administration of STZ.

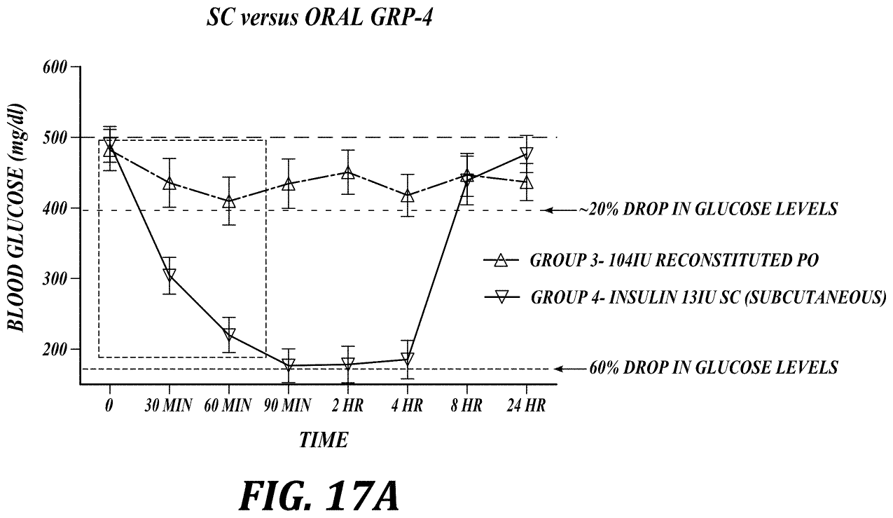

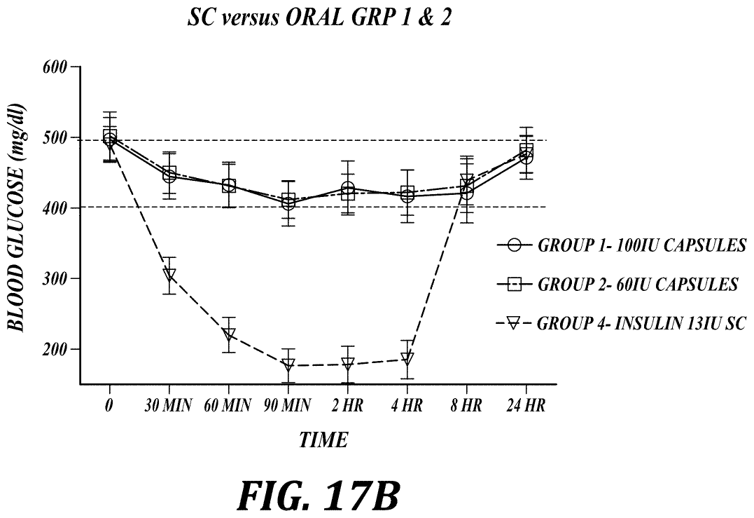

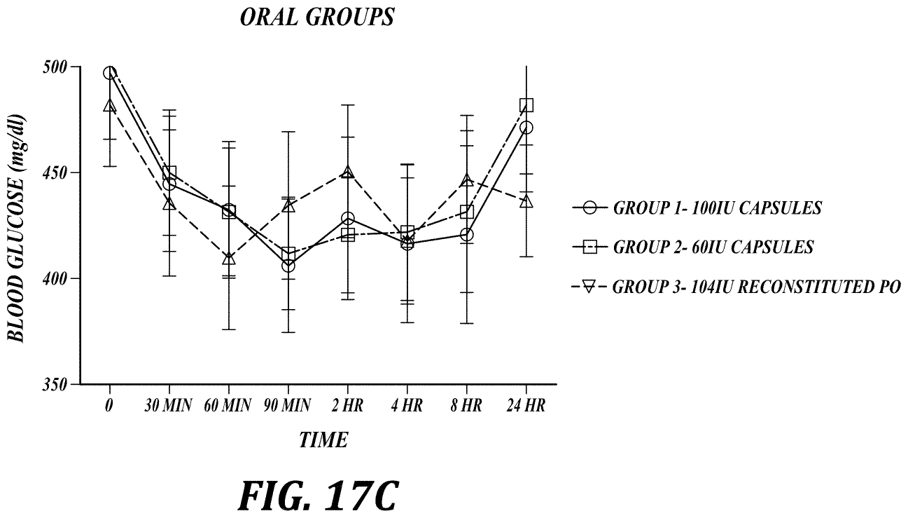

FIG. 17A graphically illustrates blood glucose levels (mg/dL) versus time in rats orally administered a pharmaceutical composition according to an embodiment of the present disclosure and subcutaneously administered insulin. In particular, FIG. 17A compares insulin levels in rats subcutaneously administered control insulin with rats administered a pharmaceutical composition according to Group 3 from TABLE 10. Similarly, FIG. 17B graphically illustrates blood glucose levels (mg/dL) versus time in rats orally administered a pharmaceutical composition according to an embodiment of the present disclosure (specifically, Groups 1 and 2 described in TABLE 10) and subcutaneous insulin. FIG. 17C graphically illustrates blood glucose levels (mg/dL) versus time in rats orally administered a pharmaceutical composition according to an embodiment of the present disclosure (Groups 1-3 of TABLE 10). As shown, rats that were orally administered a pharmaceutical composition according to Groups 1-3 had stably maintained and lowered glucose levels over greater than 8 hours.

The results from the animal study showed that for both orally administered test groups, within 1 h of administration, the animals showed glucose lowering effect >20% (compared to diabetic rodent levels), where the reduced glucose levels are maintained for >8 h. For the SC control group, although the animals showed greater glucose lowering (>60%) effect, the glucose levels rise (compared to diabetic rodent levels) within 4 h of administration. These results indicated sustained effect of the developed oral insulin formulations due to a) ion-pair complexation of insulin and b) encapsulation within HP-beta CD, both of which contributed towards gastrointestinal protection and maintaining prolonged glucose levels in diabetic rodents.

### Example 9: Insulin Stability Under Extreme Stress Conditions

The Present Example illustrates the thermostability of control insulin and Formulation C described in previous Example 3 (see TABLE 4) heated at 95° C.

Unformulated insulin and Formulation C as described further herein were reconstituted in DI water at 0.5 mg/mL.

Both samples, containing 1 ml volume were heated at 95° C.; at T=10, 30, 60 mins, samples were removed and tested for Insulin content using the HPLC method.

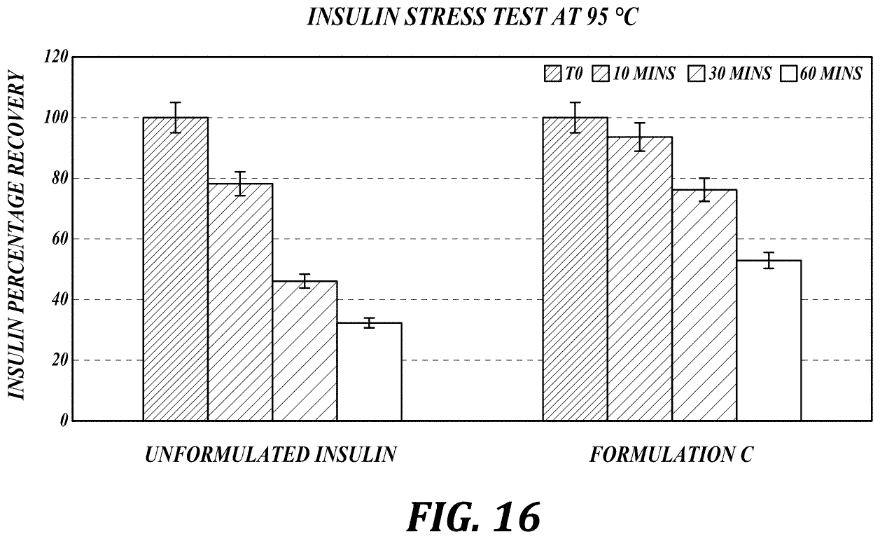

FIG. 16 graphically illustrates thermostability of an unformulated insulin and a pharmaceutical composition according to an embodiment of the present disclosure over time.

As shown in FIG. 16, the formulations according to embodiments of the present disclosure have greater thermostability over time at elevated temperatures.

### Example 10: Nucleic Acid Formulations

The present Example describes nucleic acid pharmaceutical compositions and according to embodiments of the present disclosure. See, for example, FIGS. 5A and 5B.

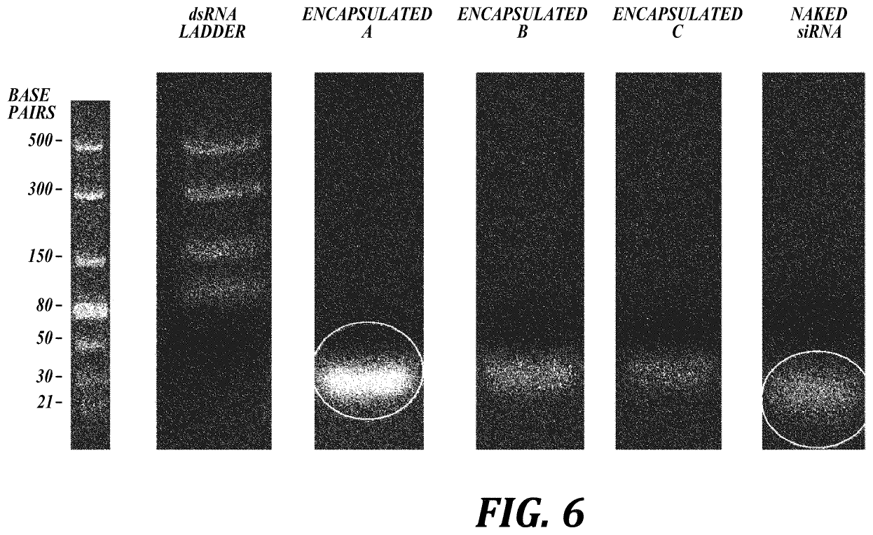

An exemplary RNA sequence, GAPDH siRNA (Glyceraldehyde-3-phosphate dehydrogenase) having 21 nucleotides, was encapsulated according to the procedure shown in FIG. 5B. Gel electrophoresis was performed to determine the formation of an encapsulated lipoplex. For example, an encapsulated lipoplex siRNA showed a shift in band size compared to naked siRNA (FIG. 6).

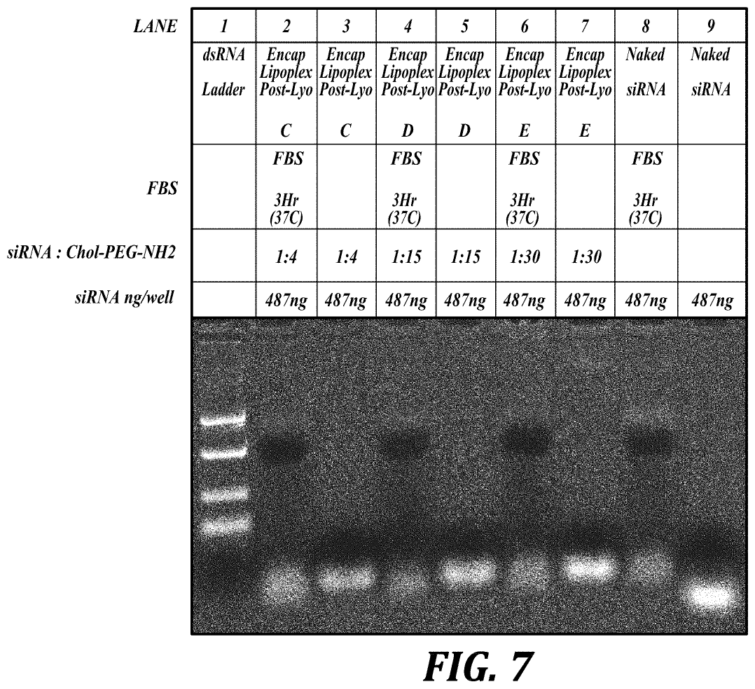

The temperature tolerance and stability against degradation by RNAse was demonstrated by incubating the encapsulated Lipoplex containing different ratios of siRNA to Chol-PEG-amine in fetal bovine serum for 3 h at 37° C. The results are shown in FIG. 7 and TABLE 11 below. As shown, the nucleic acid-based compositions described in the present Example are generally more stable than naked siRNA.

The agarose gel analysis indicated that ratio of siRNA:Chol-PEG-Amine at 1:4 encapsulated within beta cyclodextrin was most resistant to RNAse degradation maintaining >90% siRNA content based on band quantification (TABLE 11) as compared to the respective untreated Lipoplex.

TABLE 11
AGAROSE GEL BAND QUANTIFICATION RESULTS

| | | FBS | Band | FBS band Intensity/ |
|---|---|---|---|---|
| Lane | Lipoplex Mix | or Control | Intensity | Control Band Intensity |
| 2 | Post-Lyo C | FBS | 32156 | 94% |
| 3 | Post-Lyo C | Control | 34283 | |
| 4 | Post-Lyo D | FBS | 15793 | 32% |
| 5 | Post-Lyo D | Control | 49504 | |
| 6 | Post-Lyo E | FBS | 28793 | 48% |
| 7 | Post-Lyo E | Control | 60142 | |
| 8 | Naked siRNA | FBS | 29126 | 38% |
| 9 | Naked siRNA | Control | 76747 nm | |

### Definitions

At various places in the present specification, groups or ranges are described. It is specifically intended that the disclosure include each and every individual sub-combination of the members of such groups and ranges.

The verb “comprise” and its conjugations, are used in the open and non-limiting sense to mean that items following the word are included, but items not specifically mentioned are not excluded.

As used herein, the articles “a,” “an,” and “the” may include plural referents unless otherwise expressly limited to one-referent, or if it would be obvious to a skilled artisan from the context of the sentence that the article referred to a singular referent.

Where a numerical range is disclosed herein, such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include 1 and 10, and any and all subranges between the minimum value of 1 and the maximum value of 10. Exemplary subranges of the range “1 to 10” include, but are not limited to, e.g., 1 to 6.1, 3.5 to 7.8, and 5.5 to 10.

The terms “therapeutic agent”, “active agent”, “drug”, “vaccine,” and “active pharmaceutical ingredient” (API) are used interchangeably herein, and can include a small molecule, a peptide, a protein, a DNA sequence, an RNA sequence, or an mRNA sequence.

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety or a molecule that is not attracted to water with significant apolar surface area at physiological pH and/or salt conditions. This phase separation can be observed via a combination of dynamic light scattering and aqueous NMR measurements. A hydrophobic therapeutic or vaccine agent has a log P value of 1 or greater.

As used herein, the term “hydrophilic” refers to a moiety or a molecule that is attracted to and tends to be dissolved by water. The hydrophilic moiety is miscible with an aqueous phase. A hydrophilic therapeutic or vaccine agent has a log P value of less than 1.

As used herein, “lipophilic” refers to compounds that are water-insoluble with an affinity towards non-polar/hydrophobic compounds. Hydrophobic compounds can be lipophilic.

As used herein, an ionic association complex refers to a complex formed by ionic interaction between a negatively charged molecule such as a lipophilic molecule (e.g., a lipophilic detergent) with a positively charged molecule, peptide, or protein. The lipophilic ion-associate complex is insoluble in water at an acidic or a neutral pH, but dissociates at a pH of about 9 and dissolves into solution. An example of a lipophilic detergent is bile salt. An example of a positively charged molecule, peptide, or protein is insulin.

As used herein, an “inclusion complex” or an “inclusion compound” refers to a chemical complex in which one chemical compound (the “host”) has a cavity into which a “guest” compound can be accommodated. For example, the host compound can encapsulate a guest compound in its hydrophobic cavity. Without wishing to be bound by theory, it is believed that an inclusion complex formed with an hydroxypropyl-β-cyclodextrin host is held by weak Van der Waals forces.

As used herein, the term “water-insoluble” refers to a compound that has a water-solubility of less than 0.2 mg/mL (e.g., less than 0.1 mg/mL, or less than 0.01 mg/mL)), at a temperature of 25° C., and at a pressure of 1 atm or 101.3 kPa.

As used herein, the term “water-soluble” refers to a compound that is soluble in water in an amount of 1 mg/ml or more (e.g., 2 mg/ml or more), at a temperature of 25° C., and at a pressure of 1 atm or 101.3 kPa.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “polymer” refers to a macromolecule having more than 10 repeating units.

As used herein, the term “small molecule” refers to a low molecular weight (<2000 Daltons) organic compound that may help regulate a biological process, with a size on the order of 1 nm.

As used herein, “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound/combination of compounds, as well as providing directions to carry out a method involving a particular compound/combination of compounds or a dosage form comprising the compound/combination of compounds.

As used herein, a “composition” refers to a collection of materials containing the specified components. One or more dosage forms may constitute a composition, so long as those dosage forms are associated and designed for use together.

As used herein, a “pharmaceutical composition” refers to a formulation of a compound/combination of compounds of the disclosure, and a medium generally accepted in the art for the delivery of the biologically active compound to mammals, e.g., humans. Such a medium includes all pharmaceutically acceptable carriers, diluents, or excipients therefor. The pharmaceutical composition may be in various dosage forms or contain one or more unit-dose formulations. The pharmaceutical composition can provide stability over the useful life of the composition, for example, for a period of several months. The period of stability can vary depending on the intended use of the composition.

As used herein, “salts” include derivatives of an active agent, wherein the active agent is modified by making acid or base addition salts thereof. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

As used herein, a “homogeneous mixture” or “homogeneous distribution” refers to a mixture in which the components (e.g., therapeutic or vaccine agents and excipients) are uniformly distributed throughout the mixture, which can be, for example, a suspension, a powder, or a solution. The mixture can have the same physical properties at every macroscopic sampling point of the assembled drug combination product.

As used herein, an “aqueous dispersion” refers to an aqueous suspension where the therapeutic or vaccine agents and excipients of the pharmaceutical composition are suspended in a solvent or a buffer.

“Prodrug” refers to a precursor of the pharmaceutically active agent wherein the precursor itself may or may not be pharmaceutically active but, upon administration, will be converted, either metabolically or otherwise, into the active agent or drug of interest. For example, prodrug includes an ester or an ether form of an active agent.

Particular pharmacokinetic parameters are defined in TABLE 11.

TABLE 11

| Parameter | Definition |
|---|---|
| AUC_{0-t last} | Area under the plasma concentration-time curve from |
| | time zero up to the last quantifiable concentration |
| AUC_{0-∞} | Area under the plasma concentration-time curve from |
| | time zero to infinity |
| % AUCₑₓₜᵣₐₚ | Percentage of AUC that is due to extrapolation from |
| | t last to infinity |
| Cₘₐₓ | Maximum observed plasma concentration |
| tₘₐₓ | Time of the maximum observed plasma concentration |
| t_{lag} | Time before the start of absorption |
| tₗₐₛₜ | Time of the last quantifiable plasma concentration |
| t_{1/2} | Apparent plasma terminal elimination half-life |
| | (terminal half-life) |

It is noted that AUC₀₋ₜ and AUC₀₋ₜₗₐₛₜ are used interchangeably herein. Also, AUC_{inf} and AUC_{t-inf} are used interchangeably with AUC_{0-∞}. It should also be understood that, unless otherwise specified, all pharmacokinetic parameters are measured after a single administration of the specified amount of a therapeutic or vaccine agent/combination of therapeutic or vaccine agents followed by a washout period in which no additional therapeutic or vaccine agent/combination of therapeutic or vaccine agents is administered.

A “half-life” refers to the time required to divide the plasma concentration by two after reaching pseudo-equilibrium, and not the time required to eliminate half the administered dose. This is typically referred to as the last phase of descending plasma drug concentration over time and just before the drug is eliminated from the body.

A “therapeutically effective plasma concentration” refers to a plasma concentration of a therapeutic agent (i.e., drug, therapeutic agent, or vaccine composition) that elicits the biological or medicinal response that is being sought in a tissue, system, animal, individual or human by a researcher, veterinarian, medical doctor, or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition, or disorder; and

(3) ameliorating the disease; for example, ameliorating a disease, condition or disorder in an individual who is experiencing or displaying the pathology or symptomatology of the disease, condition, or disorder (i.e., reversing the pathology and/or symptomatology) such as decreasing the severity of disease.

As used herein, the phrase “therapeutically effective amount” refers to the amount of a therapeutic agent (i.e., drug, therapeutic agent, or vaccine composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition, or disorder; and

(3) ameliorating the disease; for example, ameliorating a disease, condition or disorder in an individual who is experiencing or displaying the pathology or symptomatology of the disease, condition, or disorder (i.e., reversing the pathology and/or symptomatology) such as decreasing the severity of disease.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

As used herein, the term “composite” refers to a composition material, a material made from two or more constituent materials with significantly different physical or chemical properties that, when combined, produce a material with characteristics different from the individual components. The individual components remain separate and distinct within the finished structure.

Individuals suitable for receiving these compositions depend on the nature of the therapeutic agent, as well as the disease/condition/disorder to be treated and/or prevented. Accordingly, the terms “individual” and “subject” include any of vertebrates, mammals, and humans depending on intended suitable use. In some embodiments, the individual is a mammal. In some embodiments, the individual is any one or more of human, bovine, equine, feline, canine, rodent, or primate. In some embodiments, the individual is a human. As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

Unless defined otherwise, any feature within any aspect or embodiment of the disclosure may be combined with any feature within any other aspect or embodiment of the invention, and such combination are encompassed in the present disclosure. This also applies, but not exclusively, to endpoints of ranges disclosed herein. For instance, if a given substance is dis27closed as existing in a composition in a concentration range of X-Y % or A-B %, the present disclosure is to be understood as explicitly disclosing not only the ranges X-Y % and A-B %, but also the ranges X-B %, A-Y % and, in as far as numerically possible, Y-A % and B-X %. Each of these ranges, and range combinations, are contemplated, and are to be understood as being directly and unambiguously disclosed in the present application.

Unless stated otherwise, the designation of a range in the present application using a hyphen (“-”) separating two bracketing values X and Y, or two bracketing ratios, is to be understood as meaning and disclosing the specified range in which both endpoint values X and Y are included. The same applies to a range expressed as “from X to Y”. Accordingly, the expressions of ranges as “X-Y”, “of X to Y”, “from X to Y”, “of X-Y” and “from X-Y” are to be understood equivalently as meaning and disclosing a range encompassing the end value X, all values (including decimals) between X and Y, as well as the end value Y.

As used herein the term “about” when referring to a particular value, e.g., an endpoint or endpoints of a range, encompasses and discloses, in addition to the specifically recited value itself, a certain variation around that specifically recited value. Such a variation may for example arise from normal measurement variability, e.g., in the weighing or apportioning of various substances by methods known to the skilled person. The term “about” shall be understood as encompassing and disclosing a range of variability above and below an indicated specific value, said percentage values being relative to the specific recited value itself, as follows: The term “about” may encompass and disclose variability of ±5.0%. The term “about” may encompass and disclose variability of ±4.5%. The term “about” may encompass and disclose variability of ±4.0%. The term “about” may encompass and disclose variability of ±3.5%. The term “about” may encompass and disclose variability of ±3.0%. The term “about” may encompass and disclose variability of ±2.5%. The term “about” may encompass and disclose variability of ±2.0%. The term “about” may encompass and disclose variability of ±1.5%. The term “about” may encompass and disclose variability of ±1.0%. The term “about” may encompass and disclose variability of ±0.5%. The term “about”, in reference to the particular recited value, may encompass and disclose that exact particular value itself, irrespective of any explicit mention that this exact particular value is included; even in the absence of an explicit indication that the term “about” includes the particular exact recited value, this exact particular value is still included in the range of variation created by the term “about”, and is therefore disclosed in the present application. Unless stated otherwise, where the term “about” is recited before the first endpoint of a numerical range, but not before the second endpoint of that range, this term, and the variability it implies in scope and disclosure, refers to both the first endpoint of the range and the second endpoint of the range. For instance, a recited range of “about X to Y” should be read as “about X to about Y”. The same applies for a recited range of ratios. For instance, a recited range of weight ratios of “about X:Y-A:B” should be read as a weight ratio of “(about X):(about Y)-(about A):(about B)”.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES.

While illustrative embodiments have been illustrated and described, it will be appreciated that various changes can be made therein without departing from the spirit and scope of the invention.

1. A pharmaceutical composition, comprising:
an active pharmaceutical ingredient-carrier ionic association complex, wherein the active pharmaceutical ingredient comprises insulin, wherein the carrier comprises a bile salt, wherein the bile salt comprises a deoxycholate salt, wherein the deoxycholate salt is selected from the group consisting of sodium deoxycholate, sodium cholate, and sodium taurocholate; and
a β-cyclodextrin or derivative thereof, wherein the β-cyclodextrin derivative comprises a hydroxy propyl β-cyclodextrin,
wherein the β-cyclodextrin encapsulates the active pharmaceutical ingredient-carrier ionic association complex to form a β-cyclodextrin-active pharmaceutical ingredient-carrier inclusion complex.

2. (canceled)

3. The pharmaceutical composition of claim 1, wherein the β-cyclodextrin or derivative thereof is present in the pharmaceutical composition in an amount of from about 2% to about 10% by weight.

4-10. (canceled)

11. The pharmaceutical composition of claim 1, wherein the ionic association complex comprises insulin:bile salt at a molar ratio of from 1:7 to 1:28.

12-14.

15. The pharmaceutical composition of claim 1, wherein the pharmaceutical composition has greater stability to enzymatic degradation compared to an unencapsulated active pharmaceutical ingredient ionic association complex or to a free active pharmaceutical ingredient.

16. A pharmaceutical composition, comprising:
an active pharmaceutical ingredient-cationic carrier ionic association complex; and
a β-cyclodextrin or derivative thereof,
wherein the β-cyclodextrin encapsulates the active pharmaceutical ingredient-carrier ionic association complex to form a β-cyclodextrin-active pharmaceutical ingredient-carrier inclusion complex, and
wherein the active pharmaceutical ingredient is selected from an RNA agent, a DNA agent, or a combination thereof.

17-20. (canceled)

21. The pharmaceutical composition of claim 1, wherein the pharmaceutical composition comprises an oral dosage form.

22. The pharmaceutical composition of claim 1, wherein the pharmaceutical composition comprises a solid oral dosage form, a liquid oral dosage form, or a semi-solid oral dosage form.

23. The pharmaceutical composition of claim 1, wherein the pharmaceutical composition is in the form of a tablet, a capsule, a sachet, a powder, a granule, or an orally dispersible film.

24. The pharmaceutical composition of claim 1, wherein the pharmaceutical composition is in the form of a freeze-dried tablet.

25. (canceled)

26. The pharmaceutical composition of claim 1, wherein the pharmaceutical composition comprises a suspension.

27. (canceled)

28. The pharmaceutical composition of claim 1, wherein the pharmaceutical composition has a sustained release of the active pharmaceutical ingredient at least 40% of a Cₘₐₓ in plasma over 12 hours.

29. The pharmaceutical composition of claim 1, wherein the pharmaceutical composition is stable at a pH of less than 7.

30. The pharmaceutical composition of claim 1, wherein the pharmaceutical composition is absorbable in the intestines, when ingested by a mammalian subject.

31. The pharmaceutical composition of claim 1, wherein the pharmaceutical composition is at least 90% by weight unchanged at a temperature of 30° C. and 65% relative humidity for at least 12 weeks and/or at a temperature of 40° C. and 75% relative humidity for at least 8 weeks.

32. A method of forming a pharmaceutical composition, comprising:
mixing an amount of an active pharmaceutical ingredient and an amount of a carrier in an aqueous medium to provide an active pharmaceutical ingredient-carrier ionic association complex, wherein the active pharmaceutical ingredient comprises insulin, wherein the carrier comprises a bile salt, wherein the bile salt comprises a deoxycholate salt, wherein the deoxycholate salt is selected from the group consisting of sodium deoxycholate, sodium cholate, and sodium taurocholate; and
adding a β-cyclodextrin or derivative thereof to the active pharmaceutical ingredient-carrier ionic association complex to provide a β-cyclodextrin-active pharmaceutical ingredient-carrier ionic inclusion complex.

33. (canceled)

34. (canceled)

35. A method of treating a subject having diabetes, comprising orally administrating a therapeutically effective amount of a pharmaceutical composition, the pharmaceutical composition comprising:
an active pharmaceutical ingredient-carrier ionic association complex, wherein the active pharmaceutical ingredient comprises insulin, wherein the carrier comprises a bile salt, wherein the bile salt comprises a deoxycholate salt, wherein the deoxycholate salt is selected from the group consisting of sodium deoxycholate, sodium cholate, and sodium taurocholate; and
a β-cyclodextrin or derivative thereof, wherein the β-cyclodextrin derivative comprises a hydroxy propyl β-cyclodextrin,
wherein the β-cyclodextrin encapsulates the active pharmaceutical ingredient-carrier ionic association complex to form a β-cyclodextrin-active pharmaceutical ingredient-carrier inclusion complex.

36. The pharmaceutical composition of claim 1, wherein the deoxycholate salt is sodium deoxycholate.

37. The pharmaceutical composition of claim 1, wherein the deoxycholate salt is sodium cholate.

38. The pharmaceutical composition of claim 1, wherein the deoxycholate salt is sodium taurocholate.
